(19) United States
(12) Patent Application Publication
(10) Pub. No.: US 2016/0287712 A1
(43) Pub. Date: Oct. 6, 2016

(54) FATTY ACID NIACIN CONJUGATES

(71) Applicant: CATABASIS PHARMACEUTICALS, INC., Cambridge, MA (US)

(72) Inventors: Michael R. Jirousek, Cambridge, MA (US); Jill C. Milne, Brookline, MA (US); Chi B. Vu, Boston, MA (US)

(21) Appl. No.: 15/036,106

(22) PCT Filed: Nov. 14, 2014
(86) PCT No.: PCT/US14/65810

§ 371 Date: May 12, 2016

Related U.S. Application Data
(60) Provisional application No. 61/904,929, filed on Nov. 15, 2013.

(51) Int. Cl. A61K 47/48; A61K 31/455; A61K 45/06; C07D 213/82; C07D 401/12

(52) U.S. Cl. CPC A61K 47/481; C07D 213/82; C07D 401/12; A61K 45/06; A61K 31/455

(57) ABSTRACT

The invention provides fatty acid niacin conjugates; pharmaceutical compositions comprising an effective amount of the fatty acid niacin conjugate; and methods for treating or preventing a metabolic disease comprising the administration of an effective amount of the fatty acid niacin conjugate.

### CROSS REFERENCE TO RELATED APPLICATIONS

This application claims the benefit of and priority to U.S. Provisional Patent Application Ser. No. 61/904,929, filed Nov. 15, 2013, the contents of which are hereby incorporated by reference.

### FIELD OF THE INVENTION

The invention relates to N-alkylated fatty acid niacin conjugates and their ability to preferentially accumulate in the liver as compared to other tissues, and to inhibit proprotein convertase subtilisin/kexin type 9 (PCSK9) expression and/or production preferentially in liver tissues. The invention also provides methods for treating or preventing a metabolic disease comprising the administration of N-alkylated fatty acid niacin conjugates.

### BACKGROUND OF THE INVENTION

Recent studies have demonstrated that proprotein convertase subtilisin/kexin type 9 (PCSK9) is an attractive therapeutic target for lowering low-density lipoprotein-cholesterol (LDL-C). In terms of validation, gain or loss-of-function PCSK9 variants in humans have been shown to result in hypercholesterolemia or hypocholesterolemia respectively. For instance, gain-of-function mutations in the PCSK9 gene are associated with elevated serum LDL-C levels of >300 mg/dL and premature cardiovascular heart disease (Abifadel et al Nat. Gent. 2003, 34, p. 154-156). On the other hand, loss-of-function mutations in the PCSK9 gene are associated with low serum LDL-C of ≦100 mg/dL and a reduction in cardiovascular heart disease (Cohen et al Nat. Gent. 2005, 37, p. 161-165).

PCSK9 is a serine protease, made primarily by the liver and intestine, and consists of a signal peptide, a prodomain, a catalytic domain, and the histidine-rich C terminal domain (Piper et al Structure 2007, 15, p. 545-552). Data has shown that PCSK9 can exert its effects on LDL-C by binding to hepatocyte LDL receptor and preventing it from recycling to the cell surface after endocytosis. This sequence of events results in reduced LDL receptor levels, decreased cellular uptake of LDL-C, and higher LDL-C levels in blood (Horton et al J. Lip. Res. 2009, 50 (Suppl.), p. S172-S177).

Neutralizing antibodies to PCSK9 have been shown to significantly reduce serum LDL-C in mice and nonhuman primates (Chan et al. in PNAS 2009, 106, p. 9820-9825; Liang et al. in Pharmacology and Experimental Therapeutics 2012, 340, p. 228-236). REGN727, AMG 145, RN316, and LGT209 are representative of the monoclonal antibodies that are currently being evaluated in human clinical trials for hypercholesterolemia.

Low-density lipoprotein-cholesterol is associated with numerous cardiovascular disorders. Further, despite the progress made in treating cardiovascular disorders, a large segment of the population continues to suffer from cardiovascular disorders. Thus, the need exists for new therapies for treating cardiovascular disorders and related conditions.

### SUMMARY

The invention provides N-alkylated fatty acid niacin conjugates, pharmaceutical compositions containing such conjugates, and methods of treating medical disorders using such conjugates and pharmaceutical compositions. Exemplary N-alkylated fatty acid niacin conjugates include the following compounds and pharmaceutically acceptable salts thereof:

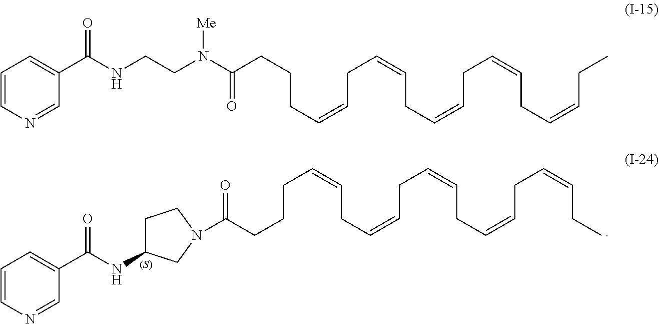

The compounds may be part of a pharmaceutical composition comprising a pharmaceutically acceptable carrier.

One surprising benefit of these N-alkylated fatty acid niacin conjugates is their ability to preferentially accumulate in the liver as compared to other tissues, and their ability to inhibit proprotein convertase subtilisin/kexin type 9 (PCSK9) expression and/or production preferentially in liver tissues. Because lipids such as cholesterol are more efficiently cleared from the plasma by the liver, compounds such as I-15 and I-24, by accumulating preferentially in the liver and blocking the activity of PCSK9, are more effective in lowering plasma cholesterol than other fatty acid conjugates linked through secondary amides. Thus, compounds I-15 and I-24 are more effective in inhibiting PCSK9 in the liver and lowering plasma cholesterol, and provide superior benefits in treating various diseases.

Accordingly, the invention provides methods for treating or preventing a metabolic disease. The method comprises administering to a patient in need thereof an effective amount of an N-alkylated fatty acid niacin conjugate described herein, such as a compound of Formula I-15, I-24, or a pharmaceutically acceptable salt thereof, to treat or prevent the disease. Exemplary metabolic diseases contemplated for treatment and/or prevention include, for example, atherosclerosis, dyslipidemia, coronary heart disease, hypercholesterolemia, cardiovascular disease, heterozygous familial hypercholesterolemia, homozygous familial hypercholesterolemia, fatty liver disease, nonalcoholic fatty liver disease (NFLD), nonalcoholic steatohepatitis (NASH), and fatty liver disease induced by treatment with a microsomal triglyceride transfer protein (MTP) inhibitor.

### BRIEF DESCRIPTION OF THE FIGURES

FIG. 1 depicts chemical structures of compounds of the invention and compounds used as comparative compounds in the Examples.

FIG. 2 is comparison of the ratio of the Portal AUCₗₐₛₜ of the Parent to the Portal AUCₗₐₛₜ of the associated Niacin-linker metabolite for compounds of the present invention. The axis on the left is the ratio of the Parent to Niacin linker ratio while the axis on the right is the absolute values for the Portal AUCₗₐₛₜ (hr*ng/ml) for the Parent and Niacin Linker for each of the compounds of the present invention.

### DETAILED DESCRIPTION OF THE INVENTION

The invention provides N-alkylated fatty acid niacin conjugates, pharmaceutical compositions containing such conjugates, and methods of treating medical disorders using such conjugates and pharmaceutical compositions. One surprising benefit of N-alkylated fatty acid niacin conjugates described herein is their ability to preferentially accumulate in the liver as compared to other tissues, and their ability to inhibit proprotein convertase subtilisin/kexin type 9 (PCSK9) expression and/or production preferentially in liver tissue. Because lipids such as cholesterol are more efficiently cleared from the plasma by the liver, compounds described herein such as I-15 and I-24, by accumulating preferentially in the liver and blocking the activity of PCSK9, are more effective in lowering plasma cholesterol than other fatty acid conjugates linked through secondary amides. Thus, compounds I-15 and I-24 are more effective in inhibiting PCSK9 in the liver and lowering plasma cholesterol, and provide superior benefits in treating various diseases such as metabolic diseases.

The practice of the present invention employs, unless otherwise indicated, conventional techniques of organic chemistry, pharmacology, cell biology, and biochemistry. Such techniques are explained in the literature, such as in “Comprehensive Organic Synthesis” (B. M. Trost & I. Fleming, eds., 1991-1992); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety. Various aspects of the invention are set forth below in sections; however, aspects of the invention described in one particular section are not to be limited to any particular section.

### I. DEFINITIONS

The following definitions are used in connection with the fatty acid niacin conjugates:

The term “fatty acid niacin conjugates” includes any and all possible isomers, stereoisomers, enantiomers, diastereomers, tautomers, pharmaceutically acceptable salts, hydrates, solvates, and prodrugs of the fatty acid derivatives described herein.

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The term “about” when used in this disclosure along with a recited value means the value recited and includes the range of + or −10% of the value. For example, the phrase about 80% means 80% and + or −10% of 80, i.e. 72% to 88%. The recited value “about 0%” as used herein means that the detectable amount is less than one part per thousand.

The term “fatty acid” as used herein means an omega-3 fatty acid and fatty acids that are metabolized in vivo to omega-3 fatty acids. Non-limiting examples of fatty acids are all-cis-7,10,13-hexadecatrienoic acid, α-linolenic acid (ALA or all-cis-9,12,15-octadecatrienoic acid), stearidonic acid (STD or all-cis-6,9,12,15-octadecatetraenoic acid), eicosatrienoic acid (ETE or all-cis-11,14,17-eicosatrienoic acid), eicosatetraenoic acid (ETA or all-cis-8,11,14,17-eicosatetraenoic acid), eicosapentaenoic acid (EPA or all-cis-5,8,11,14,17-eicosapentaenoic acid), docosapentaenoic acid (DPA, clupanodonic acid or all-cis-7,10,13,16,19-docosapentaenoic acid), docosahexaenoic acid (DHA or all-cis-4,7,10,13,16,19-docosahexaenoic acid), tetracosapentaenoic acid (all-cis-9,12,15,18,21-docosahexaenoic acid), or tetracosahexaenoic acid (nisinic acid or all-cis-6,9,12,15,18,21-tetracosenoic acid).

The term “niacin” as used herein means the molecule known as niacin and any derivative thereof.

The term “bioactive” as used herein means an aryl, including phenyl or naphthyl, heteroaryl, or a heterocycle derivative which posseses biological activity.

A “subject” is a mammal, e.g., a human, mouse, rat, guinea pig, dog, cat, horse, cow, pig, or non-human primate, such as a monkey, chimpanzee, baboon or rhesus, and the terms “subject” and “patient” are used interchangeably herein. In certain embodiments, the subject is a human.

The invention also includes pharmaceutical compositions comprising an effective amount of a fatty acid derivative of formula I-15 or I-24 as described above and a pharmaceutically acceptable carrier. The invention includes a fatty acid niacin derivative provided as a pharmaceutically acceptable prodrug, hydrate, salt, such as a pharmaceutically acceptable salt, enantiomers, stereoisomers, or mixtures thereof.

Representative “pharmaceutically acceptable salts” include, e.g., water-soluble and water-insoluble salts, such as the acetate, amsonate (4,4-diaminostilbene-2, 2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

The term “metabolic disease” as used herein refers to disorders, diseases and syndromes involving dyslipidemia, and the terms metabolic disorder, metabolic disease, and metabolic syndrome are used interchangeably herein.

An “effective amount” when used in connection with a fatty acid derivative is an amount effective for treating or preventing a metabolic disease.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body.

The term “treating”, with regard to a subject, refers to improving at least one symptom of the subject's disorder. Treating can be curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a compound or pharmaceutically acceptable salt of the compound or a composition to a subject, or administering a prodrug derivative or analog of the compound or pharmaceutically acceptable salt of the compound or composition to the subject, which can form an equivalent amount of active compound within the subject's body.

The term “prodrug,” as used in this disclosure, means a compound which is convertible in vivo by metabolic means (e.g., by hydrolysis) to a fatty acid niacin conjugate.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

### II. EXEMPLARY FATTY ACID NIACIN CONJUGATES

Fatty acid niacin conjugates of the invention are designed to be stable in plasma. However, once delivered inside cells, intracellular enzymes hydrolyze the fatty acid niacin conjugates into the individual components (i.e., niacin and omega-3 fatty acid) to produce a synergistic effect on lipid pathways not replicated by simple administration of the individual components or a combination of the individual components. One particular pathway upon which the fatty acid niacin conjugates can act synergistically is the PCSK9 axis, which is expressed primarily in the liver and intestine and which is responsible, in part, for binding to the LDL receptor, leading to its degradation and thus affecting plasma cholesterol levels.

One aspect of the invention provides the following compounds:

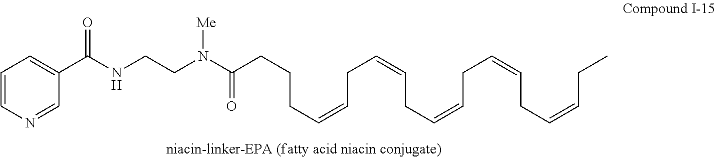 [SMILES: CC/C=C\C/C=C\C/C=C\C/C=C\C/C=C\CCCC(=O)N(C)CCNC(=O)c1cccnc1]

having chemical name N-(2-((5Z,8Z,11Z,14Z,17Z)—N-methylicosa-5,8,11,14,17-pentaenamido)ethyl)nicotinamide (I-15), and

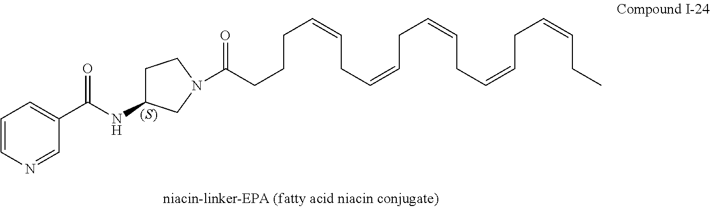

having chemical name N—((S)-1-((5Z,8Z,11Z,14Z,17Z)-icosa-5,8,11,14,17-pentaenoyl)pyrrolidin-3-yl)nicotinamide (I-24). The invention also encompasses pharmaceutically acceptable salts of the foregoing compounds.

These fatty acid niacin conjugates of the invention, while stable in the plasma, are hydrolyzed into the individual components in targeted tissues. When dosed in vivo, compound I-15 is found to be present in plasma and tissues, along with the following metabolites:

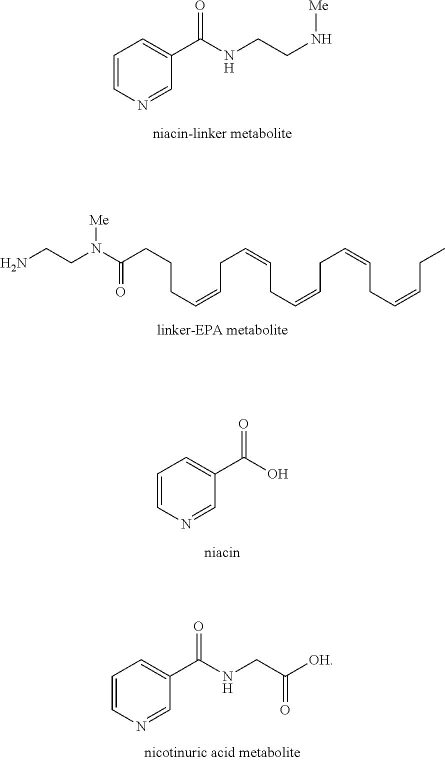 [SMILES: CNCCNC(=O)c1cccnc1 ; CC/C=C\C/C=C\C/C=C\C/C=C\C/C=C\CCCC(=O)N(C)CCN ; O=C(O)c1cccnc1 ; O=C(O)CNC(=O)c1cccnc1]

Similarly, compound I-24, when dosed in vivo, is found to be present in the plasma and tissues, along with the following metabolites:

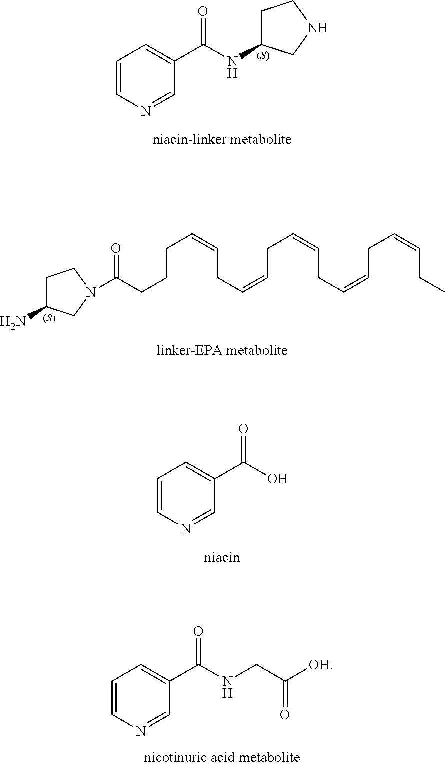

The corresponding niacin-linker metabolite is found to be generated when the amide bond between the fatty acid moiety and one end of the linker is hydrolyzed by intracellular enzymes. The corresponding linker-EPA metabolite is expected to be generated when the amide bond between the niacin portion and one end of the linker is hydrolyzed by the action of intracellular enzymes. This hydrolysis, in turn, generated niacin. Once niacin is generated intracellularly, it can undergo a further conjugation to afford the well-known nicotinuric acid metabolite.

One unexpected discovery is that compounds such as I-15 and I-24 (tertiary amide linkers) preferentially accumulate in liver tissues, e.g, in comparison to accumulation in intestinal tissues, and since lipids such as cholesterol are more efficiently cleared from the plasma by the liver, compounds such as I-15 and I-24, by accumulating preferentially in the liver and blocking the activity of PCSK9 are more effective in lowering plasma cholesterol than other fatty acid conjugates linked through secondary amides. Thus, compounds I-15 and I-24 are more effective in inhibiting PCSK9 in the liver and lowering plasma cholesterol, and present new treatments for diseases.

Another aspect of the invention provides the following compound:

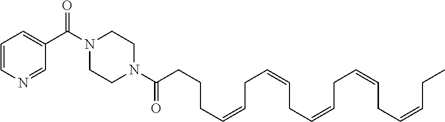 [SMILES: CC/C=C\C/C=C\C/C=C\C/C=C\C/C=C\CCCC(=O)N1CCN(C(=O)c2cccnc2)CC1]

having the chemical name (5Z,8Z,11Z,14Z,17Z)-1-(4-nicotinoylpiperazin-1-yl)icosa-5,8,11,14,17-pentaen-1-one (I-13). The invention also encompasses pharmaceutically acceptable salts of the foregoing compound.

Another aspect of the invention provides the following compound:

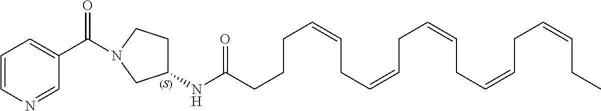

having the chemical name (5Z,8Z,11Z,14Z,17Z)—N—((S)-1-nicotinoylpyrrolidin-3-yl)icosa-5,8,11,14,17-pentaenamide (I-23). The invention also encompasses pharmaceutically acceptable salts of the foregoing compound.

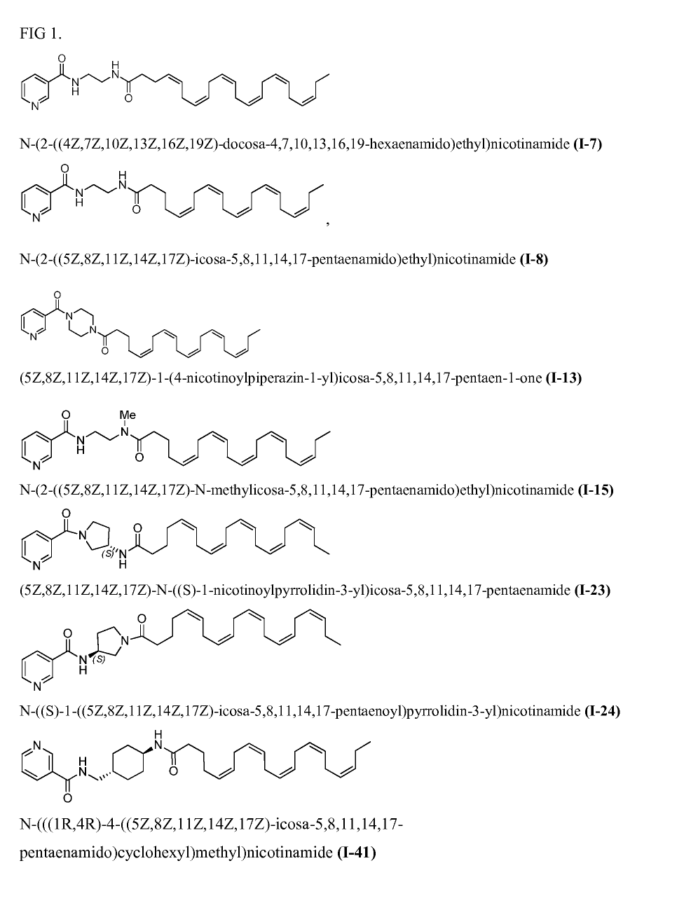

For perspective, U.S. Patent Application Publication No. 2011/0053990 describes certain fatty acid niacin conjugates, prepared by covalently linking niacin directly or indirectly to an omega-3 fatty acid such as DHA or EPA. Compounds 1-7 and 1-8 (structures shown in FIG. 1) from U.S. Patent Application Publication No. 2011/0053990 were analyzed for tissue distribution and resistance to hydrolytic degradation in Example 13 herein. These fatty acid niacin conjugates were prepared by covalently linking niacin to an omega-3 fatty acid using a variety of diamino linkers.

### III. THERAPEUTIC APPLICATIONS

Another aspect of the invention provides methods for treating or preventing a metabolic disease. The method comprises administering to a patient in need thereof an effective amount of an N-alkylated fatty acid niacin conjugate described herein, such as a compound of Formula I-15, I-24, or a pharmaceutically acceptable salt thereof, to treat or prevent the disease.

In certain embodiments, the method is to treating the metabolic disease. In other embodiments, the method is to preventing the metabolic disease.

In certain embodiments, the metabolic disease is atherosclerosis, dyslipidemia, coronary heart disease, hypercholesterolemia, cardiovascular disease, heterozygous familial hypercholesterolemia, homozygous familial hypercholesterolemia, fatty liver disease, nonalcoholic fatty liver disease (NFLD), nonalcoholic steatohepatitis (NASH), or fatty liver disease induced by treatment with a microsomal triglyceride transfer protein (MTP) inhibitor. In certain embodiments, the metabolic disease is hypertriglyceridemia, hypercholesterolemia, fatty liver disease, nonalcoholic steatohepatitis, or dyslipidemia. In certain embodiments, the metabolic disease is hypertriglyceridemia. In certain embodiments, the metabolic disease is hypercholesterimia.

In certain embodiments, the patient is treated with a statin. In certain embodiments, the patient is treated with an MTP inhibitor. In certain embodiments, the patient is treated with a Nieman Pick protein inhibitor. In certain embodiments, the Nieman Pick protein inhibitor is ezetimimide.

In certain embodiments, the N-alkylated fatty acid niacin conjugate is a compound of Formula I-15 or a pharmaceutically acceptable salt thereof. In certain embodiments, the N-alkylated fatty acid niacin conjugate is a compound of Formula I-24 or a pharmaceutically acceptable salt thereof. In certain embodiments, the N-alkylated fatty acid niacin conjugate is a compound of Formula I-13 or a pharmaceutically acceptable salt thereof. In certain embodiments, the N-alkylated fatty acid niacin conjugate is a compound of Formula I-23 or a pharmaceutically acceptable salt thereof.

In certain embodiments, the patient is a human, such as adult human.

### General Aspects

Metabolic diseases encompass a variety of medical disorders that interfere with a subject's metabolism. Metabolism is the process a subject's body uses to transform food into energy. Metabolism in a subject with a metabolic disease is disrupted in some way. The fatty acid niacin conjugates possess the ability to treat or prevent metabolic diseases. The fatty acid niacin conjugates have been designed to bring together omega-3 fatty acids and niacin into a single fatty acid bioactive derivative. The activity of the fatty acid niacin conjugates is substantially greater than the sum of the individual components of the fatty acid bioactive derivative, suggesting that the activity induced by the fatty acid niacin conjugates is synergistic.

Omega-3 fatty acids administered alone can lower triglycerides. Indeed, omega-3 fatty acids (EPA/DHA) have been shown to decrease triglycerides and to reduce the risk for sudden death caused by cardiac arrhythmias in addition to improve mortality in patients at risk of a cardiovascular event.

In view of the foregoing, a study was conducted to determine if varying the linker in the fatty acid niacin conjugates could lead to more effective conjugates in lowering the production of PCSK9 in in vitro cell assays. Such improved fatty acid niacin conjugates would possibly lower the serum PCSK9 level when dosed in vivo. In addition, a fatty niacin conjugate that accumulates preferentially in the liver, would be useful for treating hypercholesterolemia, familial heterozygous hypercholesterolemia, familial homozygous hypercholesterolemia, and fatty liver disease induced by the use of microsomal triglyceride transfer protein (MTP) inhibitors. Study results are described in the Examples herein. Due to the invention compounds' activity profile, the compounds are contemplated to be useful as a monotherapy or as a combination therapy with a statin or other cholesterol lowering agent to effectively treat hypercholesterolemia, dyslipidemia, and metabolic diseases.

### Method of Treating Metabolic Disease

One aspect of the invention provides methods for treating metabolic diseases such as the treatment or prevention of metabolic diseases including atherosclerosis, dyslipidemia, coronary heart disease, hypercholesterolemia, Type 2 diabetes, elevated cholesterol, metabolic syndrome, cardiovascular disease, heterozygous familial hypercholesterolemia, homozygous familial hypercholesterolemia, and fatty liver disease induced by treatment with a microsomal triglyceride transfer protein (MTP) inhibitor. The method comprises administering to a patient in need thereof an effective amount of a N-alkylated fatty acid niacin conjugate described herein, such as a compound of Formula I-15, I-24, or a pharmaceutically acceptable salt thereof, to treat or prevent the disease.

In one embodiment, the method involves the inhibition of PCSK9 by fatty acid derivatives. Inhibition of PCSK9 will lead to a reduction in LDL-C.

In one embodiment, the method comprises contacting a cell with a fatty acid derivative in an amount sufficient to decrease the release of triglycerides or VLDL or LDL or cause an increase in reverse cholesterol transport or increase HDL concentrations.

Another aspect of the invention provides a method for inhibiting, preventing, or treating a metabolic disease, or symptoms of a metabolic disease, in a subject. Examples of such disorders include, but are not limited to atherosclerosis, dyslipidemia, hypertriglyceridemia, hypertension, heart failure, cardiac arrhythmias, low HDL levels, high LDL levels, sudden death, stable angina, coronary heart disease, acute myocardial infarction, secondary prevention of myocardial infarction, cardiomyopathy, endocarditis, type 2 diabetes, insulin resistance, impaired glucose tolerance, hypercholesterolemia, stroke, hyperlipidemia, hyperlipoproteinemia, chronic kidney disease, intermittent claudication, hyperphosphatemia, carotid atherosclerosis, peripheral arterial disease, diabetic nephropathy, hypercholesterolemia in HIV infection, acute coronary syndrome (ACS), non-alcoholic fatty liver disease, arterial occlusive diseases, cerebral arteriosclerosis, cerebrovascular disorders, myocardial ischemia, and diabetic autonomic neuropathy.

Because of the ability of fatty acid niacin conjugates and other fatty acid conjugates used as PCSK9 inhibitors to lower cholesterol and triglycerides, they can also be used to treat diseases of the liver such as fatty liver disease, nonalcoholic fatty liver disease (NFLD), nonalcoholic steatohepatitis (NASH).

In some embodiments, the fatty acid niacin conjugates and other fatty acid conjugates used as PCSK9 inhibitors can be used to treat familial hyperlipidemia. Hyperlipidemia are classified according to which types of lipids are elevated, that is hypercholesterolemia, hypertriglyceridemia, or both in combined hyperlipidemia. Elevated levels of lipoprotein may also be classified as a form of hyperlipidemia.

There are five types of hyperlipoproteinemia (types I through V) and these are further classified according to the Fredrikson classification, based on the pattern of lipoproteins on electrophoresis or ultracentrifugation. Type I hyperlipoproteinemia has three subtypes: Type Ia (also called Buerger-Gruetz syndrome or familial hyperchylomicronemia), Type Ib (also called familial apoprotein CII deficiency) and Type Ic. Due to defects in either decreased in lipoprotein lipase (LPL), altered ApoC2 or LPL inhibitor in blood, all three subtypes of Type I hyperlipoproteinemia share the same characteristic increase in chylomicrons. The frequency of occurrence for Type I hyperlipoproteinemia is 1 in 1,000,000 and thus far treatment has consisted mainly of diet. Because of the ability of fatty acid niacin conjugates in affecting postprandial lipids, it can be especially useful in treating Type I hyperlipoproteinemia.

Type II hyperlipoproteinemia has two subtypes: Type IIa (also called familial hypercholesterolemia) is characterized by an elevated level of low-density lipoprotein (LDL); and Type IIb (also called familial combined hyperlipidemia) is characterized by an elevated level of LDL and very-low density lipoprotein (VLDL).

Type III hyperlipoproteinemia (also called familial dysbetalipoproteinemia) is characterized by an elevated level of intermediate-density lipoprotein (IDL).

Type IV hyperlipoproteinemia (also called familial hypertriglyceridemia) is characterized by an elevated level of VLDL.

Type V hyperlipoproteinemia is characterized by an elevated level of VLDL and chylomicrons. Treatment for Type V hyperlipoproteinemia thus far has not been adequate with using just niacin or fibrate. Because of the ability of fatty acid niacin conjugates in affecting postprandial lipids, it can be especially useful in treating Type V hyperlipoproteinemia.

In some embodiments, the subject is administered an effective amount of a fatty acid niacin conjugate.

### Methods for Inhibition of PCSK9

Another aspect of the invention provides a method for inhibiting the production of PCSK9 or lowering serum levels of PCSK9 in a patient. The method comprises administering to a patient in need thereof an effective amount of a N-alkylated fatty acid niacin conjugate described herein, such as a compound of formula I-15, I-24, or pharmaceutically acceptable salt thereof, to inhibit the production of PCSK9 or lower serum levels of PCSK9.

### Pharmaceutical Compositions for Medical Use

Another aspect of the invention provides a pharmaceutical composition comprising a N-alkylated fatty acid niacin conjugate as an effective ingredient having accumulation in liver tissue and/or resistance to hydrolytic degradation, wherein the N-alkylated fatty acid niacin conjugate is one of the following or a pharmaceutically acceptable salt thereof:

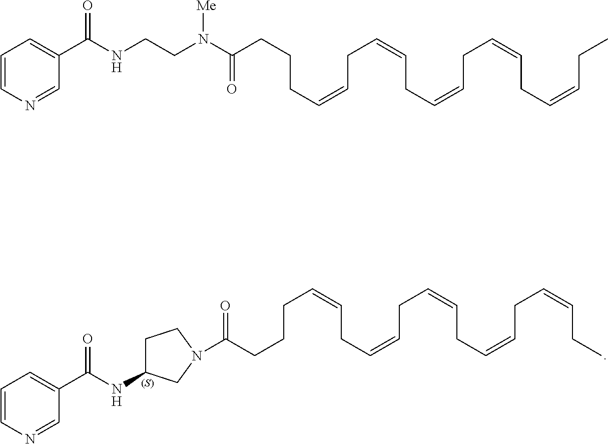

In certain embodiments, the ingredient accumulates in liver tissue. In certain embodiments, a greater amount of the ingredient accumulates in liver tissue than in intestinal tissue. In certain embodiments, the amount of the ingredient that accumulates in liver tissue is greater than two-fold, ten-fold, twenty-fold, 50-fold, or 100-fold than the amount of the ingredient that accumulates in intestinal tissue (e.g., when evaluated in a rat). In certain embodiments, the ingredient accumulates in the liver at an amount that is greater than twenty times the amount of the following compound that accumulates in the liver following oral administration of an equimolar amount of the following compound:

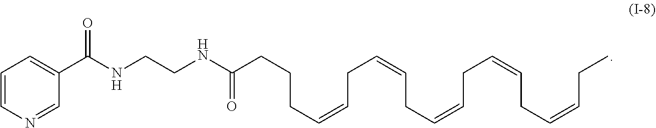 [SMILES: CC/C=C\C/C=C\C/C=C\C/C=C\C/C=C\CCCC(=O)NCCNC(=O)c1cccnc1]

In certain embodiments, the ingredient accumulates in the liver of a rat at an amount that is greater than twenty times the amount of the following compound that accumulates in the liver of a rat following oral administration of an equimolar amount of the following compound:

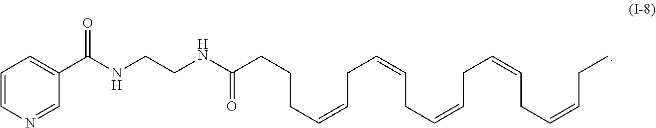 [SMILES: CC/C=C\C/C=C\C/C=C\C/C=C\C/C=C\CCCC(=O)NCCNC(=O)c1cccnc1]

In certain embodiments, the ingredient has resistance to hydrolytic degradation, such as hydrolytic degradation in vivo. In certain embodiments, the resistance to hydrolytic degradation is resistance to hydrolytic degradation in the intestine.

In certain embodiments, the ingredient accumulates in liver tissue and has resistance to hydrolytic degradation.

In certain embodiments, the N-alkylated fatty acid niacin conjugate is

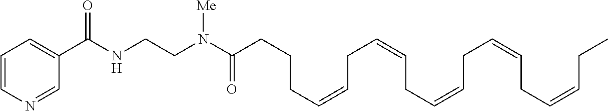 [SMILES: CC/C=C\C/C=C\C/C=C\C/C=C\C/C=C\CCCC(=O)N(C)CCNC(=O)c1cccnc1]

or a pharmaceutically acceptable salt thereof.

In certain embodiments, the N-alkylated fatty acid niacin conjugate is

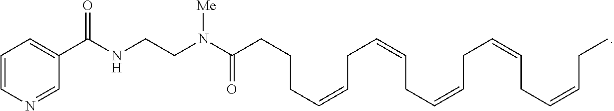 [SMILES: CC/C=C\C/C=C\C/C=C\C/C=C\C/C=C\CCCC(=O)N(C)CCNC(=O)c1cccnc1]

In certain embodiments, the N-alkylated fatty acid niacin conjugate is

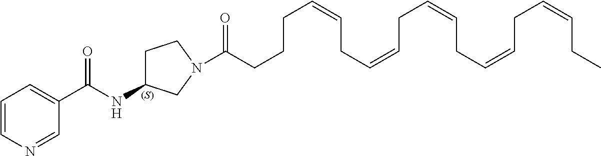

or a pharmaceutically acceptable salt thereof.

In certain embodiments, the N-alkylated fatty acid niacin conjugate is

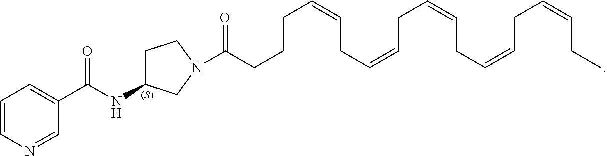

In certain embodiments, the pharmaceutical composition is for use in treating a metabolic disease. In certain embodiments, the metabolic disease is atherosclerosis, dyslipidemia, coronary heart disease, hypercholesterolemia, cardiovascular disease, heterozygous familial hypercholesterolemia, homozygous familial hypercholesterolemia, fatty liver disease, nonalcoholic fatty liver disease (NFLD), nonalcoholic steatohepatitis (NASH), or fatty liver disease induced by treatment with a microsomal triglyceride transfer protein (MTP) inhibitor. In certain embodiments, the metabolic disease is hypertriglyceridemia, hypercholesterolemia, fatty liver disease, nonalcoholic steatohepatitis, or dyslipidemia. In certain embodiments, the metabolic disease is hypertriglyceridemia.

Another aspect of the invention provides a pharmaceutical composition comprising a N-alkylated fatty acid niacin conjugate as an effective ingredient having accumulation in liver tissue and/or resistance to hydrolytic degradation, wherein the N-alkylated fatty acid niacin conjugate is the following or a pharmaceutically acceptable salt thereof:

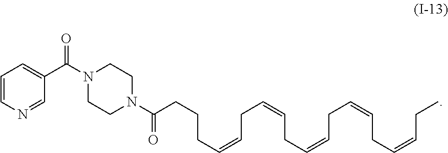 [SMILES: CC/C=C\C/C=C\C/C=C\C/C=C\C/C=C\CCCC(=O)N1CCN(C(=O)c2cccnc2)CC1]

In certain embodiments, the ingredient accumulates in liver tissue. In certain embodiments, a greater amount of the ingredient accumulates in liver tissue than in intestinal tissue. In certain embodiments, the amount of the ingredient that accumulates in liver tissue is greater than two-fold, ten-fold, twenty-fold, 50-fold, or 100-fold than the amount of the ingredient that accumulates in intestinal tissue (e.g., when evaluated in a rat). In certain embodiments, the ingredient accumulates in the liver at an amount that is greater than twenty times the amount of the following compound that accumulates in the liver following oral administration of an equimolar amount of the following compound:

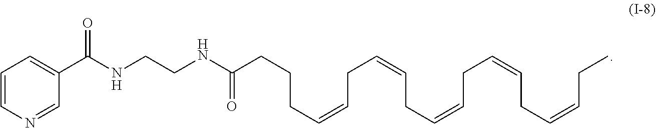 [SMILES: CC/C=C\C/C=C\C/C=C\C/C=C\C/C=C\CCCC(=O)NCCNC(=O)c1cccnc1]

In certain embodiments, the ingredient accumulates in the liver of a rat at an amount that is greater than twenty times the amount of the following compound that accumulates in the liver of a rat following oral administration of an equimolar amount of the following compound:

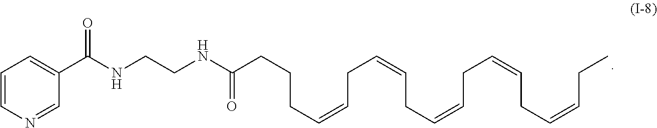 [SMILES: CC/C=C\C/C=C\C/C=C\C/C=C\C/C=C\CCCC(=O)NCCNC(=O)c1cccnc1]

In certain embodiments, the ingredient has resistance to hydrolytic degradation, such as hydrolytic degradation in vivo. In certain embodiments, the resistance to hydrolytic degradation is resistance to hydrolytic degradation in the intestine.

In certain embodiments, the ingredient accumulates in liver tissue and has resistance to hydrolytic degradation.

In certain embodiments, the N-alkylated fatty acid niacin conjugate is

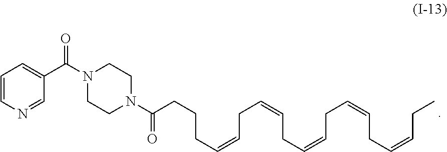 [SMILES: CC/C=C\C/C=C\C/C=C\C/C=C\C/C=C\CCCC(=O)N1CCN(C(=O)c2cccnc2)CC1]

In certain embodiments, the pharmaceutical composition is for use in treating a metabolic disease. In certain embodiments, the metabolic disease is atherosclerosis, dyslipidemia, coronary heart disease, hypercholesterolemia, cardiovascular disease, heterozygous familial hypercholesterolemia, homozygous familial hypercholesterolemia, fatty liver disease, nonalcoholic fatty liver disease (NFLD), nonalcoholic steatohepatitis (NASH), or fatty liver disease induced by treatment with a microsomal triglyceride transfer protein (MTP) inhibitor. In certain embodiments, the metabolic disease is hypertriglyceridemia, hypercholesterolemia, fatty liver disease, nonalcoholic steatohepatitis, or dyslipidemia. In certain embodiments, the metabolic disease is hypertriglyceridemia.

### Use of Compounds in the Manufacture of a Pharmaceutical Composition

Another aspect of the invention provides for use of a N-alkylated fatty acid niacin conjugate in the manufacture of a pharmaceutical composition for the treatment of a metabolic disease, the N-alkylated fatty acid niacin conjugate being an effective ingredient having accumulation in liver tissue and/or resistance to hydrolytic degradation, wherein the N-alkylated fatty acid niacin conjugate is one of the following or a pharmaceutically acceptable salt thereof:

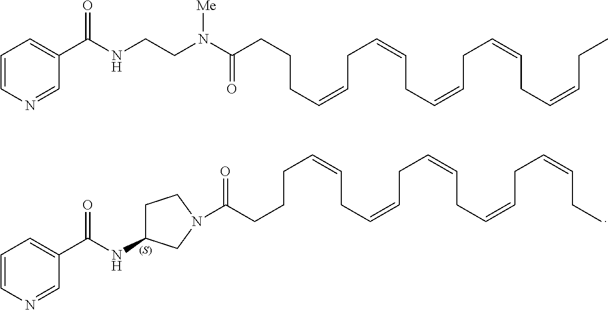

In certain embodiments, the ingredient accumulates in liver tissue. In certain embodiments, a greater amount of the ingredient accumulates in liver tissue than in intestinal tissue. In certain embodiments, the amount of the ingredient that accumulates in liver tissue is greater than two-fold, ten-fold, twenty-fold, 50-fold, or 100-fold than the amount of the ingredient that accumulates in intestinal tissue (e.g., when evaluated in a rat). In certain embodiments, the ingredient accumulates in the liver at an amount that is greater than twenty times the amount of the following compound that accumulates in the liver following oral administration of an equimolar amount of the following compound:

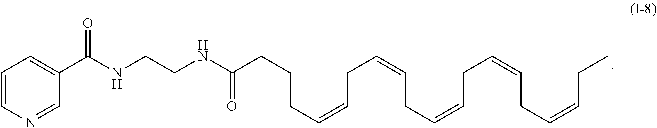 [SMILES: CC/C=C\C/C=C\C/C=C\C/C=C\C/C=C\CCCC(=O)NCCNC(=O)c1cccnc1]

In certain embodiments, the ingredient accumulates in the liver of a rat at an amount that is greater than twenty times the amount of the following compound that accumulates in the liver of a rat following oral administration of an equimolar amount of the following compound:

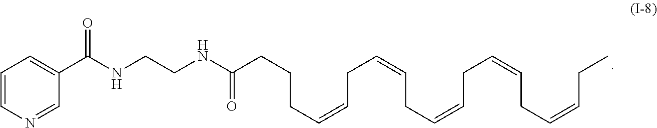 [SMILES: CC/C=C\C/C=C\C/C=C\C/C=C\C/C=C\CCCC(=O)NCCNC(=O)c1cccnc1]

In certain embodiments, the ingredient has resistance to hydrolytic degradation, such as hydrolytic degradation in vivo. In certain embodiments, the resistance to hydrolytic degradation is resistance to hydrolytic degradation in the intestine.

In certain embodiments, the ingredient accumulates in liver tissue and has resistance to hydrolytic degradation.

In certain embodiments, the N-alkylated fatty acid niacin conjugate is

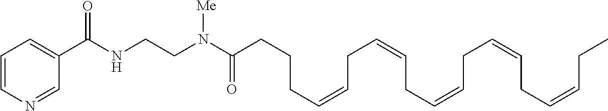 [SMILES: CC/C=C\C/C=C\C/C=C\C/C=C\C/C=C\CCCC(=O)N(C)CCNC(=O)c1cccnc1]

or a pharmaceutically acceptable salt thereof.

In certain embodiments, the N-alkylated fatty acid niacin conjugate is

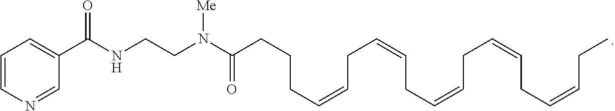 [SMILES: CC/C=C\C/C=C\C/C=C\C/C=C\C/C=C\CCCC(=O)N(C)CCNC(=O)c1cccnc1]

In certain embodiments, the N-alkylated fatty acid niacin conjugate is

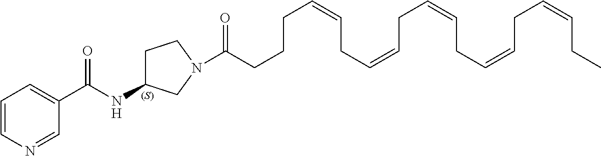

or a pharmaceutically acceptable salt thereof.

In certain embodiments, the N-alkylated fatty acid niacin conjugate is

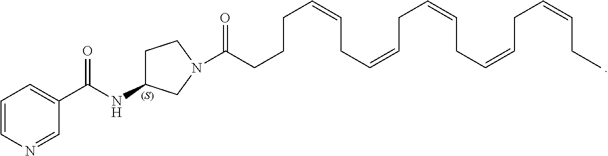

In certain embodiments, the metabolic disease is atherosclerosis, dyslipidemia, coronary heart disease, hypercholesterolemia, cardiovascular disease, heterozygous familial hypercholesterolemia, homozygous familial hypercholesterolemia, fatty liver disease, nonalcoholic fatty liver disease (NFLD), nonalcoholic steatohepatitis (NASH), or fatty liver disease induced by treatment with a microsomal triglyceride transfer protein (MTP) inhibitor. In certain embodiments, the metabolic disease is hypertriglyceridemia, hypercholesterolemia, fatty liver disease, nonalcoholic steatohepatitis, or dyslipidemia. In certain embodiments, the metabolic disease is hypertriglyceridemia.

Another aspect of the invention provides for use of a N-alkylated fatty acid niacin conjugate in the manufacture of a pharmaceutical composition for the treatment of a metabolic disease, the N-alkylated fatty acid niacin conjugate being an effective ingredient having accumulation in liver tissue and/or resistance to hydrolytic degradation, wherein the N-alkylated fatty acid niacin conjugate is the following or a pharmaceutically acceptable salt thereof:

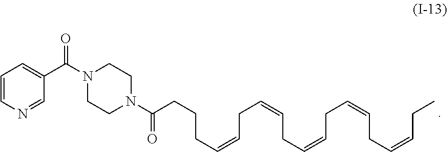 [SMILES: CC/C=C\C/C=C\C/C=C\C/C=C\C/C=C\CCCC(=O)N1CCN(C(=O)c2cccnc2)CC1]

In certain embodiments, the ingredient accumulates in liver tissue. In certain embodiments, a greater amount of the ingredient accumulates in liver tissue than in intestinal tissue. In certain embodiments, the amount of the ingredient that accumulates in liver tissue is greater than two-fold, ten-fold, twenty-fold, 50-fold, or 100-fold than the amount of the ingredient that accumulates in intestinal tissue (e.g., when evaluated in a rat). In certain embodiments, the ingredient accumulates in the liver at an amount that is greater than twenty times the amount of the following compound that accumulates in the liver following oral administration of an equimolar amount of the following compound:

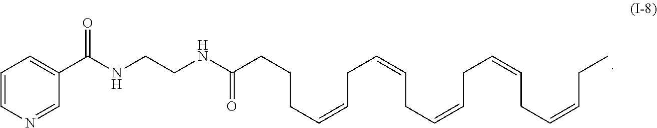 [SMILES: CC/C=C\C/C=C\C/C=C\C/C=C\C/C=C\CCCC(=O)NCCNC(=O)c1cccnc1]

In certain embodiments, the ingredient accumulates in the liver of a rat at an amount that is greater than twenty times the amount of the following compound that accumulates in the liver of a rat following oral administration of an equimolar amount of the following compound:

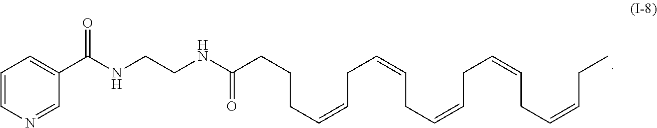 [SMILES: CC/C=C\C/C=C\C/C=C\C/C=C\C/C=C\CCCC(=O)NCCNC(=O)c1cccnc1]

In certain embodiments, the ingredient has resistance to hydrolytic degradation, such as hydrolytic degradation in vivo. In certain embodiments, the resistance to hydrolytic degradation is resistance to hydrolytic degradation in the intestine.

In certain embodiments, the ingredient accumulates in liver tissue and has resistance to hydrolytic degradation.

In certain embodiments, the N-alkylated fatty acid niacin conjugate is

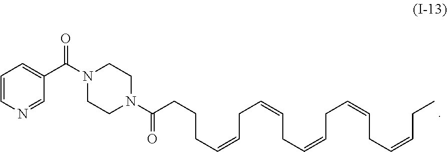 [SMILES: CC/C=C\C/C=C\C/C=C\C/C=C\C/C=C\CCCC(=O)N1CCN(C(=O)c2cccnc2)CC1]

In certain embodiments, the metabolic disease is atherosclerosis, dyslipidemia, coronary heart disease, hypercholesterolemia, cardiovascular disease, heterozygous familial hypercholesterolemia, homozygous familial hypercholesterolemia, fatty liver disease, nonalcoholic fatty liver disease (NFLD), nonalcoholic steatohepatitis (NASH), or fatty liver disease induced by treatment with a microsomal triglyceride transfer protein (MTP) inhibitor. In certain embodiments, the metabolic disease is hypertriglyceridemia, hypercholesterolemia, fatty liver disease, nonalcoholic steatohepatitis, or dyslipidemia. In certain embodiments, the metabolic disease is hypertriglyceridemia.

### IV. PHARMACEUTICAL COMPOSITIONS

The invention provides pharmaceutical compositions comprising a pharmaceutically acceptable carrier and a fatty acid niacin conjugate described herein, such as a compound of Formula I-15, I-24, or a pharmaceutically acceptable salt thereof. In certain other embodiments, the fatty acid niacin conjugate is a compound of Formula I-13, I-23, or a pharmaceutically acceptable salt thereof.

The invention also includes pharmaceutical compositions useful for treating or preventing a metabolic disease, or for inhibiting a metabolic disease, or more than one of these activities. The compositions can be suitable for internal use and comprise an effective amount of a fatty acid derivative and a pharmaceutically acceptable carrier. The fatty acid derivatives are especially useful in that they demonstrate very low peripheral toxicity or no peripheral toxicity.

Depending on the intended mode of administration, the compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, all using forms well known to those skilled in the pharmaceutical arts.

Illustrative pharmaceutical compositions are tablets and gelatin capsules comprising a fatty acid niacin derivative and a pharmaceutically acceptable carrier, such as: a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, alginic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, the fatty acid niacin derivative is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the fatty acid niacin derivatives.

The fatty acid niacin conjugates can be also formulated as a suppository that can be prepared from fatty emulsions or suspensions; using polyalkylene glycols such as propylene glycol, as the carrier.

The fatty acid niacin conjugates can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564, the contents of which are herein incorporated by reference in their entirety.

Fatty acid niacin conjugates can also be delivered by the use of monoclonal antibodies as individual carriers to which the fatty acid derivatives are coupled. The fatty acid derivatives can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the fatty acid derivatives can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels. In one embodiment, fatty acid derivatives are not covalently bound to a polymer, e.g., a polycarboxylic acid polymer, or a polyacrylate.

Parenteral injectable administration is generally used for subcutaneous, intramuscular or intravenous injections and infusions. Injectables can be prepared in conventional forms, either as liquid solutions or suspensions or solid forms suitable for dissolving in liquid prior to injection.

Compositions can be prepared according to conventional mixing, granulating or coating methods, respectively, and the present pharmaceutical compositions can contain from about 0.1% to about 90%, from about 10% to about 90%, or from about 30% to about 90% of the fatty acid derivative by weight or volume.

### V. EXEMPLARY DOSING AMOUNTS AND FEATURES

The fatty acid niacin conjugates can each be administered in amounts that are sufficient to treat or prevent a metabolic disease or prevent the development thereof in subjects.

Administration of the fatty acid niacin conjugates can be accomplished via any mode of administration for therapeutic agents. These modes include systemic or local administration such as oral, nasal, parenteral, transdermal, subcutaneous, vaginal, buccal, rectal or topical administration modes.

The dosage regimen utilizing the fatty acid derivative is selected in accordance with a variety of factors including type, species, age, weight, sex and medical condition of the patient; the severity of the condition to be treated; the route of administration; the renal or hepatic function of the patient; and the particular fatty acid niacin derivative employed. A physician or veterinarian of ordinary skill in the art can readily determine and prescribe the effective amount of the drug required to prevent, counter or arrest the progress of the condition.

Effective dosage amounts of the present invention, when used for the indicated effects, range from about 20 mg to about 5,000 mg of the fatty acid niacin conjugate per day. Compositions for in vivo or in vitro use can contain about 20, 50, 75, 100, 150, 250, 500, 750, 1,000, 1,250, 2,500, 3,500, or 5,000 mg of the fatty acid derivative, or from about one value to about any other value from the forgoing list of fatty acid niacin conjugate amounts, e.g, from about 100 mg to about 1000 mg. In one embodiment, the compositions are in the form of a tablet that can be scored.

In an embodiment, the measurements of blood plasma levels are known to the person of ordinary skill in the art, for example, standard pharmacokinetic (PK) parameters may be used to denote the amount of compound that is present in the plasma: Cₘₐₓ (maximum concentration, measured in ng/mL) and AUCₗₐₛₜ (area under the curve, measured over time). When blood/plasma samples are collected from a subject portal vein catheter, the Cₘₐₓ and AUCₗₐₛₜ may be denoted as portal Cₘₐₓ and portal AUCₗₐₛₜ. When blood/plasma samples are collected from a subject jugular vein catheter, the Cₘₐₓ and AUCₗₐₛₜ may be denoted as peripheral Cₘₐₓ and peripheral AUCₗₐₛₜ.

In an embodiment, portal Cₘₐₓ plasma levels of the fatty acid niacin conjugate can range from about 1000 ng/mL to about 20000 ng/mL, of from about 2000 to about 10000 ng/mL, or from about 2000 to about 8000 ng/mL or from about 2000 to about 6000 ng/mL.

In an embodiment the portal AUCₗₐₛₜ plasma levels of the fatty acid niacin conjugate may be from about 1000 hr*ng/mL to about 20000 hr*ng/mL, of from about 2000 to about 10000 hr*ng/mL, or from about 2000 to about 8000 hr*ng/mL or from about 2000 to about 5000 hr*ng/mL.

In an embodiment, peripheral Cₘₐₓ plasma levels of the fatty acid niacin conjugate can range from about 50 ng/mL to about 1000 ng/mL, of from about 100 to about 1000 ng/mL, or from about 100 to about 500 ng/mL or from about 250 to about 500 ng/mL.

In an embodiment the peripheral AUCₗₐₛₜ may be from about 100 hr*ng/mL to about 1000 hr*ng/mL, of from about 200 to about 1000 hr*ng/mL, or from about 400 to about 1000 hr*ng/mL. Appropriate dosages of the fatty acid niacin conjugates can be determined as set forth in Goodman, L. S.; Gilman, A. The Pharmacological Basis of Therapeutics, 5th ed.; MacMillan: New York, 1975, pp. 201-226.

Fatty acid niacin conjugates can be administered in a single daily dose, or the total daily dosage can be administered in divided doses of two, three or four times daily. Furthermore, fatty acid niacin conjugates can be administered in intranasal form via topical use of suitable intranasal vehicles, or via transdermal routes, using those forms of transdermal skin patches well known to those of ordinary skill in that art. To be administered in the form of a transdermal delivery system, the dosage administration can be continuous rather than intermittent throughout the dosage regimen. Other illustrative topical preparations include creams, ointments, lotions, aerosol sprays and gels, wherein the concentration of the fatty acid derivative ranges from about 0.1% to about 15%, w/w or w/v.

### VI. EXEMPLARY COMBINATION THERAPIES

Fatty acid niacin conjugates may be administered with other therapeutic agents such as cholesterol-lowering agents, fibrates and hypolipidemic agents, anti-diabetic agents, agents used to treat NASH and NAFLD, lipid-lowering agents and antihypertensive agents.

In some embodiments, the other therapeutic agent is a cholesterol-lowering agents. Non limiting examples of cholesterol-lowering agents are atorvastatin, cerivastatin, fluvastatin, lovastatin, pitavastatin, pravastatin, rosuvastatin, simvastatin, ezetimibe, and the combination of ezetimibe/simvastatin (Vytorin®). The statin drug class has been used extensively in the clinic to lower cholesterol. Indeed, statin treatment has been shown to significantly increase the expression of PCSK9 and secretion of PCSK9 (Dubuc et al Arterioscler. Thromb. Vasc. 2004, p. 1453-1459). The increased level of PCSK9 could essentially counteract some of the beneficial effects of statins since PCSK9 has been demonstrated to degrade LDL receptors, leading to higher plasma levels of LDC-C. In addition, co-administration of a statin along with a PCSK9 inhibitor could potentially result in a more significant reduction in LDL-C since the statin family of drugs has been shown to increase the plasma levels of PCSK9.

In some embodiments, the other therapeutic agent is a fibrate or hypolipidemic agent. Non-limiting examples of fibrates or hypolipidemic agents are acifran, acipimox, beclobrate, bezafibrate, binifibrate, ciprofibrate, clofibrate, colesevelam, gemfibrozil, fenofibrate, melinamide, and ronafibrate.

In some embodiments, the other therapeutic agent is an agent that can lower PCSK9 (proprotein convertase subtilisin/kexin type 9). Non-limiting examples include a PCSK9 monoclonal antibody such as REGN727, AMG 145, RN316, and LGT209, a biologic agent, a small interfering RNA (siRNA) and a gene silencing oligonucleotide.

In some embodiments, the other therapeutic agent is a microsomal triglyceride transfer protein (MTP) inhibitor. Non-limiting examples of MTP inhibitors include lomitapide, implitapide, CP-346086, SLx-4090, and AS1552133.

In some embodiments, the other therapeutic agent is one that can be used to treat NASH or NAFLD. Non-limiting examples of agents that can be used to treat NASH or NAFLD include cysteamine, and an FXR (farnesoid X receptor) agonist such as obeticholic acid (a bile acid analog).

In some embodiments, the other therapeutic agent is an apolipoprotein B synthesis inhibitor. Non-limiting examples of apolipoprotein B synthesis inhibitors include mipomersen, a biologic agent, a small interfering RNA (siRNA) and a gene silencing oligonucleotide.

In some embodiments, the other therapeutic agent is a CETP (cholesteryl transfer protein) inhibitor. Non-limiting examples of CETP inhibitors include dalcetrapib, evacetrapib, anacetrapib and torcetrapib.

In some embodiments, the other therapeutic agent is a lipid lowering agent. Non-limiting examples of lipid lowering agents include agents that raise ApoA-I, HM74a agonists, squalene synthetase inhibitors, and lipoprotein-associated phospholipase A2 inhibitors.

In some embodiments, the other therapeutic agent is an anti-diabetic agent. Non-limiting examples of anti-diabetic agents are acarbose, epalrestat, exenatide, glimepiride, liraglutide, metformin, miglitol, mitiglinide, nateglinide, pioglitazone, pramlintide, repaglinide, rosiglitazone, tolrestat, troglitazone, and voglibose.

In some embodiments, the other therapeutic agent is a DPP-IV (dipeptidyl peptidase-4) inhibitor as anti-diabetic agent. Non-limiting examples of DPP-IV inhibitors as anti-diabetic agents are sitagliptin, saxagliptin, vildagliptin, linagliptin, dutogliptin, gemigliptin and alogliptin.

In some embodiments, the other therapeutic agent is an antihypertensive agents. Non-limiting examples of antihypertensive agents include alacepril, alfuzosin, aliskiren, amlodipine besylate, amosulalol, aranidipine, arotinolol HCl, azelnidipine, barnidipine hydrochloride, benazepril hydrochloride, benidipine hydrochloride, betaxolol HCl, bevantolol HCl, bisoprolol fumarate, bopindolol, bosentan, budralazine, bunazosin HCl, candesartan cilexetil, captopril, carvedilol, celiprolol HCl, cicletanine, cilazapril, cinildipine, clevidipine, delapril, dilevalol, doxazosin mesylate, efonidipine, enalapril maleate, enalaprilat, eplerenone, eprosartan, felodipine, fenoldopam mesylate, fosinopril sodium, guanadrel sulfate, imidapril HCl, irbesartan, isradipine, ketanserin, lacidipine, lercanidipine, lisinopril, losartan, manidipine hydrochloride, mebefradil hydrochloride, moxonidine, nebivolol, nilvadipine, nipradilol, nisoldipine, olmesartan medoxomil, perindopril, pinacidil, quinapril, ramipril, rilmedidine, spirapril HCl, telmisartan, temocarpil, terazosin HCl, tertatolol HCl, tiamenidine HCl, tilisolol hydrochloride, trandolapril, treprostinil sodium, trimazosin HCl, valsartan, and zofenopril calcium.

Fatty acid niacin conjugates may also be administered with other therapeutic agents such as cholesterol-lowering agents, fibrates and hypolipidemic agents, anti-diabetic agents, anti-diabetic agents, antihypertensive agents and anti-inflammatory agents.

In some embodiments, the other therapeutic agent is a cholesterol-lowering agents. Non limiting examples of cholesterol-lowering agents are atorvastatin, cerivastatin, fluvastatin, lovastatin, pitavastatin, pravastatin, rosuvastatin, simvastatin, ezetimibe, and the combination of ezetimibe/simvastatin (Vytorin®).

In some embodiments, the other therapeutic agent is a fibrate or hypolipidemic agent. Non-limiting examples of fibrates or hypolipidemic agents are acifran, acipimox, beclobrate, bezafibrate, binifibrate, ciprofibrate, clofibrate, colesevelam, gemfibrozil, fenofibrate, melinamide, niacin, and ronafibrate.

Niacin at high dose (1.5 to 4 grams per day) has been shown to improve very low-density lipoprotein (“VLDL”) levels through lowering Apolipoprotein B (“ApoB”) and raising high density lipoprotein (“HDL”) through increasing Apolipoprotein A1 (“ApoA1”) in the liver. Niacin can also inhibit diacylglycerol acyltransferase-2, a key enzyme for TG synthesis (Kamanna, V. S.; Kashyap, M. L. Am. J. Cardiol. 2008, 101 (8A), 20B-26B).

In some embodiments, the other therapeutic agent is a DPP-IV inhibitor as anti-diabetic agent. Non-limiting examples of DPP-IV inhibitors as anti-diabetic agents are sitagliptin, saxagliptin, vildagliptin, linagliptin, dutogliptin, gemigliptin and alogliptin.

In some embodiments, the other therapeutic agent is an anti-diabetic agent. Non-limiting examples of anti-diabetic agents are acarbose, epalrestat, exenatide, glimepiride, liraglutide, metformin, miglitol, mitiglinide, nateglinide, pioglitazone, pramlintide, repaglinide, rosiglitazone, tolrestat, troglitazone, and voglibose.

In some embodiments, the other therapeutic agent is an antihypertensive agents. Non-limiting examples of antihypertensive agents include alacepril, alfuzosin, aliskiren, amlodipine besylate, amosulalol, aranidipine, arotinolol HCl, azelnidipine, barnidipine hydrochloride, benazepril hydrochloride, benidipine hydrochloride, betaxolol HCl, bevantolol HCl, bisoprolol fumarate, bopindolol, bosentan, budralazine, bunazosin HCl, candesartan cilexetil, captopril, carvedilol, celiprolol HCl, cicletanine, cilazapril, cinildipine, clevidipine, delapril, dilevalol, doxazosin mesylate, efonidipine, enalapril maleate, enalaprilat, eplerenone, eprosartan, felodipine, fenoldopam mesylate, fosinopril sodium, guanadrel sulfate, imidapril HCl, irbesartan, isradipine, ketanserin, lacidipine, lercanidipine, lisinopril, losartan, manidipine hydrochloride, mebefradil hydrochloride, moxonidine, nebivolol, nilvadipine, nipradilol, nisoldipine, olmesartan medoxomil, perindopril, pinacidil, quinapril, ramipril, rilmedidine, spirapril HCl, telmisartan, temocarpil, terazosin HCl, tertatolol HCl, tiamenidine HCl, tilisolol hydrochloride, trandolapril, treprostinil sodium, trimazosin HCl, valsartan, and zofenopril calcium.

In other embodiments, suitable angiotensin-converting-enzyme (ACE) inhibitors used in the above-described combination therapies include, without limitation, enalapril, ramipril, quinapril, perindopril, lisinopril, imidapril, zofenopril, trandolapril, fosinopril, and captopril.

### EXAMPLES

The invention now being generally described, will be more readily understood by reference to the following examples, which are included merely for purposes of illustration of certain aspects and embodiments of the present invention, and are not intended to limit the invention.

### Example 1

### Preparation of N-(2-(4Z,7Z,10Z,13Z,16Z,19Z)-docosa-4,7,10,13,16,19-hexaenamidoethyl)nicotinamide (I-7)

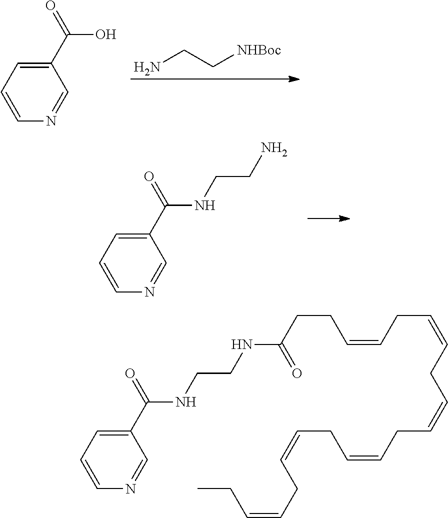 [SMILES: O=C(O)c1cccnc1 ; CC(C)(C)OC(=O)NCCN ; NCCNC(=O)c1cccnc1 ; CC/C=C\C/C=C\C/C=C\C/C=C\C/C=C\C/C=C\CCC(=O)NCCNC(=O)c1cccnc1]

In a typical run, nicotinic acid (2.0 g, 16.2 mmol) was taken up in CH₂Cl₂ (20 mL) along with oxalyl chloride (1.4 mL, 16.2 mmol). After a few drops of DMF were added, the reaction mixture was stirred at room temperature until all the solids had dissolved and all gas evolution had ceased (1 h). This freshly prepared solution of the acid chloride was added dropwise at 0° C. to a solution containing tert-butyl 2-aminoethylcarbamate (2.6 g, 16.2 mmol) and Et₃N (3.4 mL, 24.2 mmol) in CH₂Cl₂ (200 mL). The resulting reaction mixture was warmed to room temperature and stirred for 2 h. It was then washed with brine, dried over Na₂SO₄, filtered and concentrated under reduced pressure. Purification by silica gel chromatography (CH₂Cl₂) afforded tert-butyl 2-(nicotinamido)ethylcarbamate (3.1 g, 74%).

tert-Butyl 2-(nicotinamido)ethylcarbamate (3.1 g, 11.7 mmol) was taken up in 25% TFA in CH₂Cl₂ (10 mL). The resulting reaction mixture was allowed to stand at room temperature for 1 h. At this point, a considerable amount of precipitate formed and the clear filtrate was removed. The remaining solids were dried to afford of the TFA salt of N-(2-aminoethyl)nicotinamide (1.6 g).

The TFA salt of N-(2-aminoethyl)nicotinamide (5.0 mmol) was taken up in CH₃CN (20 mL) along with (4Z,7Z,10Z,13Z,16Z,19Z)-docosa-4,7,10,13,16,19-hexaenoic acid (5.0 mmol), HATU (5.5 mmol) and DIEA (15 mmol). The resulting reaction mixture was stirred at room temperature for 2 h and diluted with EtOAc. The organic layer was washed with saturated aqueous NaHCO₃, brine, dried over Na₂SO₄, filtered and concentrated under reduced pressure. Purification by silica gel chromatography (5% MeOH—CH₂Cl₂) afforded N-(2-(4Z,7Z,10Z,13Z,16Z,19Z)-docosa-4,7,10,13,16,19-hexaenamidoethyl)nicotinamide. MS calculated for C₃₀H₄₁N₃O₂: 475.32. found: [M+H]⁺ 476.

### Example 2

### Preparation of N-(2-(5Z,8Z,11Z,14Z,17Z)-eicosa-5,8,11,14,17-pentaenamidoethyl)nicotinamide (I-8)

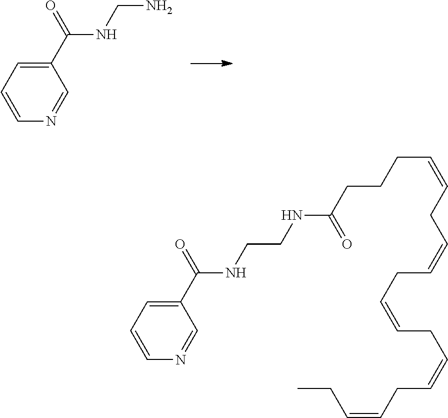 [SMILES: NCNC(=O)c1cccnc1 ; CC/C=C\C/C=C\C/C=C\C/C=C\C/C=C\CCCC(=O)NCCNC(=O)c1cccnc1]

The TFA salt of N-(2-aminoethyl)nicotinamide (1.6 g, 5.7 mmol) was taken up in CH₃CN (15 mL) along with (5Z,8Z,11Z,14Z,17Z)-eicosa-5,8,11,14,17-pentaenoic acid (1.7 g, 5.7 mmol), HATU (2.4 g, 6.3 mmol) and DIEA (3 mL, 17 mmol). The resulting reaction mixture was stirred at room temperature for 2 h and diluted with EtOAc. The organic layer was washed with saturated aqueous NaHCO₃, brine, dried over Na₂SO₄, filtered and concentrated under reduced pressure. Purification by silica gel chromatography (5% MeOH—CH₂Cl₂) afforded N-(2-(5Z,8Z,11Z,14Z,17Z)-eicosa-5,8,11,14,17-pentaenamidoethyl)nicotinamide (1.6 g, 62%). MS calculated for C₂₈H₃₉N₃O₂: 449.3. found: [M+H]⁺ 450.

### Example 3

### Preparation of N-(2-((5Z,8Z,11Z,14Z,17Z)—N-methylicosa-5,8,11,14,17-pentaenamido)ethyl)nicotinamide (I-15)

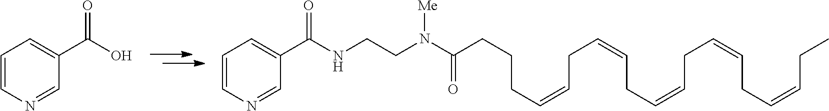 [SMILES: O=C(O)c1cccnc1 ; CC/C=C\C/C=C\C/C=C\C/C=C\C/C=C\CCCC(=O)N(C)CCNC(=O)c1cccnc1]

N-(2-((5Z,8Z,11Z,14Z,17Z)—N-Methylicosa-5,8,11,14,17-pentaenamido)ethyl)nicotinamide was prepared according to the procedures outlined in example 1, substituting the commercially available tert-butyl (2-aminoethyl)(methyl)carbamate for the diamine and EPA for the fatty acid component. MS calculated for C₂₉H₄₁N₃O₂: 463.32. found: [M+H]⁺ 464.

### Example 4

### Preparation of N—((S)-1-((5Z,8Z,11Z,14Z,17Z)-Icosa-5,8,11,14,17-pentaenoyl)pyrrolidin-3-yl)nicotinamide (I-24)

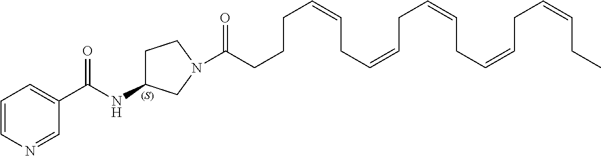

The title compound was prepared according to the same procedures outlined for compound (I-15), using the BOC-protected diamine, namely (S)-tert-butyl 3-aminopyrrolidine-1-carboxylate. MS calculated for C₃₀H₄₁N₃O₂ 475.32. found 476.

### Example 5

### Preparation of (5Z,8Z,11Z,14Z,17Z)-1-(4-nicotinoylpiperazin-1-yl)icosa-5,8,11,14,17-pentaen-1-one (I-13)

The title compound was prepared according to the same procedures outlined for compound I-15, using the BOC-protected diamine.

### Example 6

### Preparation of (5Z,8Z,11Z,14Z,17Z)—N—((S)-1-nicotinoylpyrrolidin-3-yl)icosa-5,8,11,14,17-pentaenamide (I-23)

The title compound was prepared according to the same procedures outlined for compound I-15, using the BOC-protected diamine.

### Example 7

### Preparation of N-(((1R,4R)-4-((5Z,8Z,11Z,14Z,17Z)-icosa-5,8,11,14,17-pentaenamido)cyclohexyl)methyl)nicotinamide (I-41)

The title compound was prepared according to the same procedures outlined for compound I-15, using the BOC-protected diamine.

### Example 8

### Analysis of Effect of Fatty Acid Derivatives on ApoA1 and ApoB Secretion in HepG2 Cells

The effect that compounds have on ApoA1 and ApoB Secretion in HepG2 cells can be evaluated using the procedure described below. For perspective, niacin has been reported to increase serum levels of HDL to LDL cholesterol in vivo. Similarly, niacin has been reported to increase the secretion of ApoA1 (Jin, F-Y. et al. Arterioscler. Thromb. Vasc. Biol. 1997, 17 (10), 2020-2028) while inhibiting the secretion of ApoB (Jin, F-Y. et al. Arterioscler. Thromb. Vasc. Biol. 1999, 19, 1051-1059) in the media supernatants of HepG2 cultures. Independently, DHA has been demonstrated to lower ApoB as well (Pan, M. et al. J. Clin. Invest. 2004, 113, 1277-1287) by a very different mechanism. Thus, the secretion of ApoA1 and ApoB from HepG2 cells possesses utility as a cell based read-out for niacin-DHA derivative small molecules.

HepG2 cells (ATCC) are seeded at 10,000 cells per well in 96 well plates. After adhering overnight, growth media (10% FBS in DMEM) is removed and cells are serum starved for 24 hours in DMEM containing 0.1% fatty acid free bovine serum albumin (Sigma). Cells are then treated with the compounds at 6 concentrations (2 fold dilutions starting at 100 μM). Niacin at 1.5 mM is used as a positive control. All treatments are performed in triplicate.

Simultaneous with compound treatment, ApoB secretion is stimulated with addition of 0.1 oleate complexed to fatty acid free BSA in a 5:1 molar ratio. Incubation with compounds and oleate is conducted for 24 hours. Media supernatants are removed and ApoA1 and ApoB concentrations are measured using ELISA kits (Mabtech AB).

ApoA1 is expressed as a percent increase over vehicle (0.1% ethanol) treated wells. Percent inhibition of ApoB secretion is determined by normalizing data to vehicle treated wells. For a given compound, an IC₅₀ (concentration at which 50% of ApoB secretion is inhibited) is determined by using a 4 parameter-fit inhibition curve model (Graph Pad Prism®). In each experiment, cell viability is determined using the ATPlite 1-Step kit (Perkin Elmer), such that compound effects due to cytotoxicity can be monitored.

### Example 9

### Analysis of Effect of Compounds in a PCSK9 Assay

The effect that compounds have on PCSK9 activity was evaluated using the assay described below. Experimental procedures are described in Part I. Results are described in Part II.

### Part I—Procedures

### Cell Culture

HepG2 cells (from ATCC, Catalog no. HB-8065) were maintained in DMEM (Invitrogen) supplemented with 10% fetal bovine serum (Invitrogen). The day prior to the PCSK9 assay, cells are seeded in 96-well collagen coated plates at 25,000 cells/well.

### Compound Preparation

Compounds were stored at −20° C. until used. The test article compound was dissolved in 100% ethanol to a 50 mM stock solution. This was then diluted in FBS to a final concentration of 1 mM. This solution was placed in a sonicating water bath for 30 minutes. Subsequent dilutions were then made in FBS supplemented with an equivalent volume of ethanol and mixed by vortexing.

### PCSK9 Secretion Assay

HepG2 cells were seeded onto a collagen coated 96-well plate (Becton Dickinson, Catalog no. 35-4407) the day prior to the assay, as described above. The next day, the cell medium was removed, washed once with 100 μL serum free DMEM to remove any residual PCSK9, and replaced with 90 μL of serum free DMEM. Ten microliters of each compound concentration prepared in FBS was then added. Each concentration of compound was tested in triplicate. The compound was incubated with the cells overnight for 16 hours. Following this incubation, 10 μL of AlamarBlue was added to each well and cells incubated for another 2 hours. The plates were then removed and AlamarBlue fluorescence was measured (excitation, 550 nm and excitation, 590 nm) to assess cell viability.

Cell culture supernatant was then diluted 1:5 in 1:5 in 1×RD5P Calibrator Diluent and PCSK9 ELISA was then performed with 50 μL of this diluted sample, as per the manufacturer's instructions. The ELISA was measured on a Victor X5 multilabel plate reader (PerkinElmer) at an absorbance of 450 nm with background correction measured at 550 nm (The PCSK9 Elisa kits can be purchased from R&D System, Catalog no. DPC900). To those familiar in the art, an IC50 could also be obtained when this type of assay was carried out using at least 6 different concentrations of the test compounds.

### Part II—Results

Table 1 lists compounds tested for PCSK9 inhibition activity. The assay results showed that all compounds in Table 1 had an IC50 value of <50 μM.

TABLE 1
Compounds Tested for PCSK9 Activity
Compound number

I-7
I-8
I-13
I-15
I-23
I-24
I-41

### Example 10

### Analysis of Effect of Compounds on Plasma Triglyceride Level of the Zucker fa/fa Rat Model of Dyslipidemia

The effect that compounds have on plasma triglyceride levels in the Zucker fa/fa rat model of dyslipidemia can be evaluated using the procedure described below.

Male Zucker rats (HsdHlr: Zucker-Lepr̂fa) between 8-10 weeks of age are purchased from Harlan and maintained on Purina Rodent Diet (5001) for the duration of the study. Animals are randomized and assigned to treatment arms based on body weight and plasma triglyceride (TG) levels (n=8). Inclusion criteria for the study include body weight>300 grams and fed TG levels in plasma>800 mg/dL. Dosing is initiated on day 1 and continues through day 5. Dosing is daily (qd) by oral gavage (po) for all treatment arms (e.g., Compound 1-8 is administered orally at 4 different doses, 10, 30, 100 and 300 mg/kg; in addition, a combination of niacin/EPA in a ratio of 100/200 mg/kg may be employed). Body weights are measured for all rats on days 1 through 5. On day 4, a blood sample (fed) is collected from each rat, processed for plasma and stored at −80° C. Plasma triglyceride level in the blood sample is determined using commercial kits employing standard protocols.

### Example 11

### Analysis of the Effect of a Combination of a Compound of the Invention and Atorvastatin on Plasma Cholesterol and Other Lipids in ApoE3Leiden Mice

The effect that compounds and atorvastatin have on plasma cholesterol and other lipids in ApoE3Leiden mice can be evaluated using the procedure described below.

The study uses female APOE*3Leiden mice (groups of each n=10) and one untreated reference control group on chow (n=5). To induce dyslipidemia, a high cholesterol Western type diet containing 1% cholesterol, 15% cacao butter, 40.5% sucrose and 1% corn oil (WTD) is fed to the mice for a total experimental period of 20 weeks (of which 4 weeks are a run-in period). To prevent oxidation of the test compound, 30 mg/kg alpha-tocopherol is added to the high cholesterol diets, i.e., also in the high cholesterol diet control.

In the first 4 weeks (run-in period), a pro-atherogenic state of dyslipidemia characterized by elevated plasma cholesterol levels (about 15-20 mM) is induced in all mice by feeding them an atherogenic diet containing 1% cholesterol. The mice are then separated into a control group (no treatment) and three treatment groups: i) compound of the invention, ii) atorvastatin and iii) compound of the invention+atorvastatin as described below. The dyslipidemic mice are grouped on the basis of plasma cholesterol at t=0 assayed in 4 h fasting blood. Mice with low cholesterol after the run-in period are excluded so that homogenous experimental groups are obtained. A group of reference mice (n=5) remains on a chow diet during the complete study period (normolipidemic reference mice).

Doses of test compounds may be as follows:
Compound of the invention: 0.75% w/w in diet.
Atorvastatin: 0.0015% w/w in diet (to achieve about 20% reduction in plasma cholesterol).
Alpha-tocopherol: 0.0030% w/w in diet.

The test compounds, sufficient for approximately 3 kg of diet (i.e., 25 g of compound of the invention), and alpha-tocopherol (>200 mg) are formulated before the start of the treatment period (t=0), by adding the test compounds to melted, hand warm cocoa butter and mixed for 5 min. This mix is then added to the master mix (containing the rest of the ingredients) and mixed thoroughly. The diet is frozen to −20° C. On the subsequent day, the diet is broken into small pellets (approximately 5 g per piece) and freeze dried, and stored in vacuum sealed bags (approximately 500 g) at −20° C. until use. The diets are refreshed daily and unused diet is discarded.

The following parameters are taken at the indicated time-points (individually unless mentioned otherwise):
1) Body weight at −4, 0, 2, 4 weeks
2) Food intake (g/day/mouse) at 0, 2, 4 weeks (per cage)
3) Plasma total cholesterol at −4, 0, 2, 4 weeks (individually)
4) Plasma triglycerides at −4, 0, 2, 4 weeks (individually)
5) Lipoprotein profiles at 0 (pool of all animals) and 4 weeks (cholesterol distribution over VLDL, LDL and HDL-sized particles, analysis on group level).

EDTA plasma is collected in weeks −4, 0, 2 and 4 weeks. Plasma cholesterol, plasma triglyceride levels and lipoprotein profiles are assayed immediately in fresh plasma.

### Example 12

### Analysis of Pharmacokinetics of Fatty Acid Niacin Conjugates Following Oral Administration to Male Sprague-Dawley Rats with Jugular Vein Catheter and Portal Vein Catheter

Pharmacokinetics of exemplary compounds depicted in FIG. 1 were evaluated following oral administration to Male Sprague-Dawley rats. Experimental procedures and results are provided below.

### Part I—Procedures

Pharmacokinetic studies were conducted in male Sprague-Dawley rats, with an approximate weight of 250-300 g at dose initiation. All animals were instrumented with a jugular vein catheter (JVC) and a portal vein catheter (PVC) to facilitate blood collections. Jugular and Portal vein catheterizations were conducted at Charles River Laboratories under protocols approved by the Institutional Animal Care and Use Committee (IACUC) of Charles River Lab (Approved PO 4122010).

### Surgical Procedure for Jugular Vein Cannulation

Animals were anaesthetized with ketamine and xylazine administered intraperitoneally and provided buprenorphine subcutaneously. The skin overlying the scapula and over the right jugular vein were shaved and skin aseptically prepared. A 1.1.5 cm cranial-caudal incision, followed by blunt dissection was performed to expose the jugular vein, which was then catheterized using either a PU or blended catheter. After fixation of the catheter, it was flushed with sterile saline to verify patency. The distal end of the catheter was then tunneled subcutaneously to the dorsal scapular region where it was exteriorized. Catheter patency was verified again. The catheter was then locked with 50% heparinized dextrose solution. The exteriorized end of the catheter was sealed with a stainless steel plug. The skin incision was then closed with wound clips.

### Surgical Procedure for Portal Vein Cannulation

Animals were anaesthetized with ketamine and xylazine administered intraperitoneally. A mid-line incision 1-2 cm was made in the abdominal cavity and the portal vein was detached near the liver. To prevent bleeding, the portal vein was ligated temporarily as the catheter was inserted. The cathether (3.5 Fr polyurethane tube, Access™ technologies Inc.) was inserted immediately and fixed by a purse-string suture on the portal vein. The time to reperfusion was about 1 min after intercepted blood flow. This method for insertion of catheter can avoid the occlusion of the vessel. In addition, a catheter with trumpet-shaped opening was used to prevent the catheter from slipping out of the vessel with minimizing the effect of blood flow. Another end of the catheter was passed subcutaneously to the dorsal back of the neck and the laparotomy was closed in two layers, with a 4/0 silk blade to the muscle, and a surgical clip to close the skin.

### Pharmacokinetic Study

Male Sprague-Dawley rats instrumented with jugular vein catheter and portal vein catheter were separated into groups of 4. The test compound was administered by oral gavage at dose of 30 mg/kg in a vehicle consisting of Tween (specifically Tween 80), Peceol, PEG400 (at a ratio of 40:50:10) along with 0.2% tocopherol and emulsified in water, with a water to oil ratio of 66:1 (the dose concentration was 6 mg/mL and the dose volume was 5 mL/kg). Whole blood (0.3 mL) was collected at both the jugular and portal vein at the following time points: 10, 20, 40 min, 1, 1.5, 2 and 4 hours post-dose. Blood samples were placed into tubes containing sodium heparin anticoagulant. Each whole blood sample was placed on ice until sample could be centrifuged at 2200×g at 5° C.±3° C. for 10 min to isolate plasma.

The amount of the various metabolites and parent compound present in the plasma samples were then analyzed by LC/MS/MS using an Agilent 6410 Triple Quadrupole with an ESI interface. The column used was a Phenomenex Gemini C6 Phenyl 110A 50×4.6 mm, 1.8 μM. The mobile phase used was water and methanol, each containing 0.1% formic acid. Data was further analyzed by WinNolin Software. For a given test compound, the following compounds were quantitated from the collected plasma samples: 1) test compound, 2) niacin linker, 3) linker-EPA, 4) niacin and 5) nicotinuric acid. The structures and the amount of compound that was quantitated from LC/MS/MS are tabulated in Tables 2-8.

In order to quantitate each of these compounds in plasma, standard curves in blank plasma were generated using the appropriate internal standard (the final concentration range used were 8333 ng/mL, 2083 ng/mL, 521 ng/mL, 130.2 ng/mL, 32.5 ng/mL, 8.1 ng/mL, 2.0 ng/mL and 0.5 ng/mL).

To those familiar in the art, standard pharmacokinetic (PK) parameters were used to denote the amount of compound that was present in the plasma: Cₘₐₓ (maximum concentration, measured in ng/mL) and AUCₗₐₛₜ (area under the curve, measured from t=10 min to 4 hrs). When blood/plasma samples were collected from the portal vein catheter, the Cₘₐₓ and AUCₗₐₛₜ were further denoted as portal Cₘₐₓ and portal AUCₗₐₛₜ. When blood/plasma samples were collected from the jugular vein catheter, the Cₘₐₓ and AUCₗₐₛₜ were further denoted as peripheral Cₘₐₓ and peripheral AUCₗₐₛₜ.

### Part II—Results

Compounds I-15 and I-24 provided an unexpected and preferential accumulation of corresponding parent compound (i.e., un-metabolized compound) in the liver versus the intestine. This unexpected property is reflected in the portal Cₘₐₓ and portal AUCₗₐₛₜ of the corresponding parent compound. For compound I-15, the portal Cₘₐₓ for the parent compound was 4,520 ng/mL and portal AUCₗₐₛₜ for the parent compound was 5,059 hr*ng/mL. For compound I-24, the portal Cₘₐₓ for the parent compound was 5,315 ng/mL and portal AUCₗₐₛₜ for the parent compound was 6,641 hr*ng/mL. The portal Cₘₐₓ and AUCₗₐₛₜ represent, e.g., are indicative of, the concentration of the parent compound that was being delivered to the liver. Thus, a higher portal Cₘₐₓ and AUCₗₐₛₜ values correspond to a higher concentration of the compound that is being delivered to the liver.

The unexpected and preferential accumulation of the parent compound in the liver for compounds I-15 and I-24 was evident when compared directly with a representative analog such as compound I-8. Compound I-8, with an ethylenediamine linker, had a significantly lower portal Cₘₐₓ and AUCₗₐₛₜ for the parent compound (portal Cₘₐₓ=756 ng/mL, portal AUCₗₐₛₜ=662 hr*ng/mL).

In addition, when compared with both compounds I-15 and I-24, the concentration of the metabolites niacin-linker and nicotinuric acid for compound I-8 was significantly higher. For compound I-8, the portal Cₘₐₓ for the niacin-linker metabolite was 9,253 ng/mL and the portal Cₘₐₓ for nicotinuric acid was 1,047 ng/mL. This is in sharp contrast with compound I-15 where a much lower concentration of the metabolites niacin-linker and nicotinuric acid was detected in the portal plasma (for I-15, portal Cₘₐₓ for the niacin-linker moiety was just 12 ng/mL and the portal Cₘₐₓ for nicotinuric acid was 14 ng/mL; similarly, for I-24, portal Cₘₐₓ for the niacin linker was 73 ng/mL and the portal Cₘₐₓ for nicotinuric acid was 20 ng/mL). This indicates that compound I-8 is significantly hydrolyzed in the intestine. Compounds I-7, I-13, I-23 and I-41 all showed lower portal concentration of the parent compound when compared to compounds I-15 and I-24.

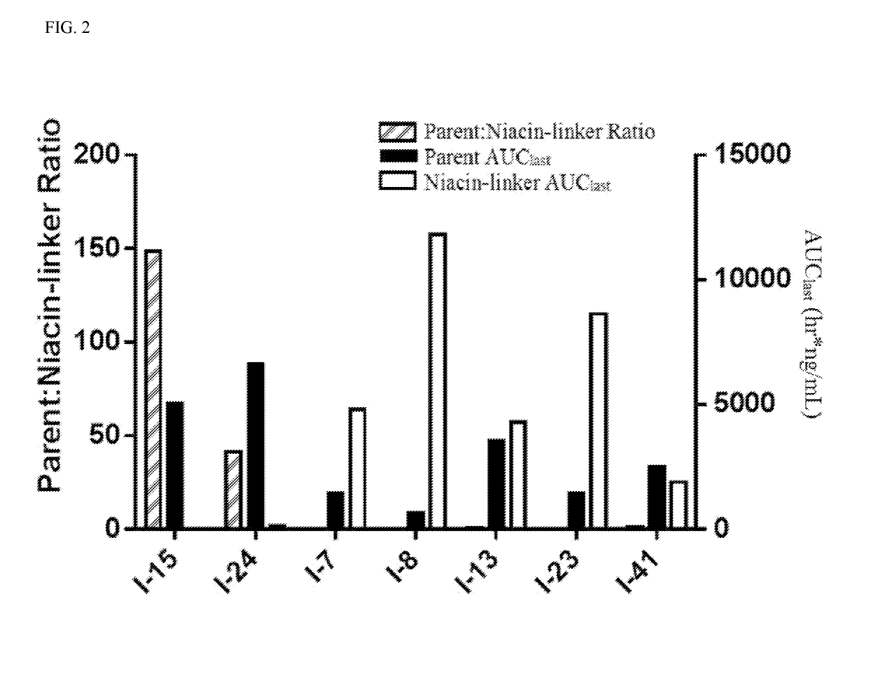

The data in Tables 2-8 were further analyzed by comparing the ratio of the Portal AUCₗₐₛₜ of each parent compound to the Portal AUCₗₐₛₜ of the associated Niacin-linker metabolite. The representative Niacin-linker metabolite is produced upon cleavage of the fatty acid (EPA or DHA)-linker bond in the parent compound. As shown in FIG. 2, compounds I-15 and I-24 have a ratio of Parent compound to Niacin-linker metabolite of 149 and 41 respectively, while the same ratio for compounds I-7, I-8, I-13, I-23 and I-41 are 0.30, 0.06, 0.83, 0.17 and 1.3, respectively.

Because more of the parent compound is being delivered to the liver, compounds I-15 and I-24 are more effective in lowering plasma cholesterol when used as a monotherapy or as a combination therapy with other cholesterol-lowering agent.

TABLE 2
Tables 2 through 8. PK Data from Jugular and Portal Vein Cannulated Sprague-Dawley rats.

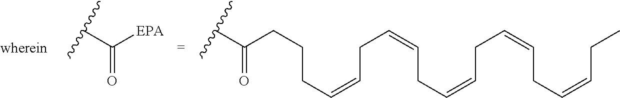

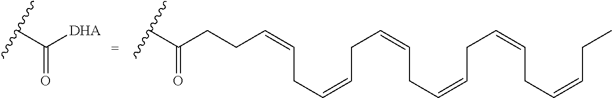

| | Niacin-linker | | | Nicotinuric |
|---|---|---|---|---|
| | Metabolite | Linker-EPA | Niacin | Acid |
| Compound I-15 | 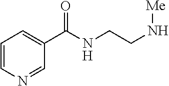 [SMILES: CNCCNC(=O)c1cccnc1] | Metabolite 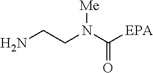 | 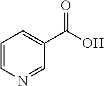 | 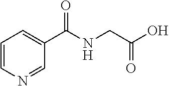 [SMILES: O=C(O)CNC(=O)c1cccnc1] |
| Portal Cₘₐₓ = | Portal Cₘₐₓ = | Portal Cₘₐₓ = | Portal Cₘₐₓ = | Portal Cₘₐₓ = |
| 4.520 ng/mL | 28 ng/mL | 12 ng/mL | below detection. | 14 ng/mL |
| Portal AUCₗₐₛₜ = | Portal AUCₗₐₛₜ = | Portal AUCₗₐₛₜ = | Portal AUCₗₐₛₜ = | Portal AUCₗₐₛₜ = |
| 5.059 hr*ng/mL | 34 hr*ng/mL | 31 hr*ng/mL | below detection | 38 hr*ng/mL |
| Peripheral Cₘₐₓ = | Peripheral Cₘₐₓ = | Peripheral Cₘₐₓ = | Peripheral Cₘₐₓ = | Peripheral Cₘₐₓ = |
| 360 ng/mL | below detection | 1.4 ng/mL | below detection | 28 ng/mL |
| Peripheral AUCₗₐₛₜ = | Peripheral AUCₗₐₛₜ = | Peripheral AUCₗₐₛₜ = | Peripheral | Peripheral AUCₗₐₛₜ = |
| 767 hr*ng/mL | below detection | 3 hr*ng/mL | AUCₗₐₛₜ = below | 29 hr*ng/mL |
| | | | detection | |

TABLE 3

| Compound I-24 | Niacin-linker Metabolite 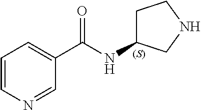 | Linker-EPA Metabolite 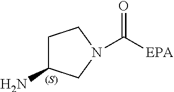 | Niacin 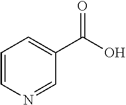 | Nicotinuric acid 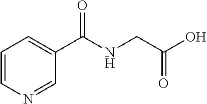 [SMILES: O=C(O)CNC(=O)c1cccnc1] |
|---|---|---|---|---|
| Portal Cₘₐₓ = | Portal Cₘₐₓ = 73 | Portal Cₘₐₓ = | Portal Cₘₐₓ = 47 | Portal Cₘₐₓ = 13 |
| 5,315 ng/mL | ng/mL | below detection | ng/mL. | ng/mL |
| Portal AUCₗₐₛₜ = | Portal AUCₗₐₛₜ = | Portal AUCₗₐₛₜ = | Portal AUCₗₐₛₜ = | Portal AUCₗₐₛₜ = |
| 6,641 hr*ng/mL | 161 hr*ng/mL | below detection | 87 hr*ng/mL | 33 hr*ng/mL |
| Peripheral Cₘₐₓ = | Peripheral Cₘₐₓ = 35 | Peripheral Cₘₐₓ = | Peripheral Cₘₐₓ = | Peripheral Cₘₐₓ = |
| 565 ng/mL | ng/mL | below detection | 3 ng/mL | 20 ng/mL |
| Peripheral AUCₗₐₛₜ = | Peripheral AUCₗₐₛₜ = | Peripheral AUCₗₐₛₜ = | Peripheral | Peripheral AUCₗₐₛₜ = |
| 964 hr*ng/mL | 76 hr*ng/mL | below detection | AUCₗₐₛₜ = 2 | 54 hr*ng/mL |
| | | | hr*ng/mL | |

TABLE 4

| Compound I-7 | Niacin-linker Metabolite 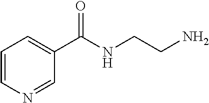 [SMILES: NCCNC(=O)c1cccnc1] | Linker-EPA Metabolite 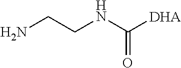 | Niacin 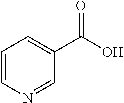 | Nicotinuric acid 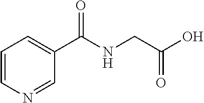 [SMILES: O=C(O)CNC(=O)c1cccnc1] |
|---|---|---|---|---|
| Portal Cₘₐₓ = | Portal Cₘₐₓ = 3270 | Portal Cₘₐₓ = | Portal Cₘₐₓ = | Portal Cₘₐₓ = 364 |
| 1,847 ng/mL | ng/mL | below detection | below detection. | ng/mL |
| Portal AUCₗₐₛₜ = | Portal AUCₗₐₛₜ = | Portal AUCₗₐₛₜ = | Portal AUCₗₐₛₜ = | Portal AUCₗₐₛₜ = |
| 1,458 hr*ng/mL | 4813 hr*ng/mL | below detection | below detection | 783 hr*ng/mL |
| Peripheral Cₘₐₓ = | Peripheral Cₘₐₓ = | Peripheral Cₘₐₓ = | Peripheral Cₘₐₓ = | Peripheral Cₘₐₓ = |
| 238 ng/mL | 938 ng/mL | below detection | below detection | 296 ng/mL |
| Peripheral | Peripheral AUCₗₐₛₜ = | Peripheral AUCₗₐₛₜ = | Peripheral | Peripheral AUCₗₐₛₜ = |
| AUCₗₐₛₜ = | 1,596 hr*ng/mL | below detection | AUCₗₐₛₜ = below | 699 hr ng/mL |
| 353 hr*ng/mL | | | detection | |

TABLE 5

| Compound I-8 | Niacin-linker Metabolite 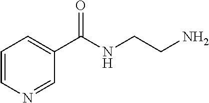 [SMILES: NCCNC(=O)c1cccnc1] | Linker-EPA Metabolite 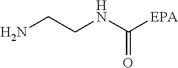 | Niacin 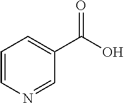 | Nicotinuric acid 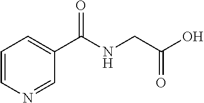 [SMILES: O=C(O)CNC(=O)c1cccnc1] |
|---|---|---|---|---|
| Portal Cₘₐₓ = 756 | Portal Cₘₐₓ = 9,253 | Portal Cₘₐₓ = | Portal Cₘₐₓ = 3 | Portal Cₘₐₓ = |
| ng/mL | ng/mL | below detection | ng/mL. | 1,047 ng/mL |
| Portal AUCₗₐₛₜ = | Portal AUCₗₐₛₜ = | Portal AUCₗₐₛₜ = | Portal AUCₗₐₛₜ = | Portal AUCₗₐₛₜ = |
| 662 hr*ng/mL | 11,818 hr*ng/mL | below detection | 2 hr*ng/mL | 3,537 hr*ng/mL |
| Peripheral Cₘₐₓ = | Peripheral Cₘₐₓ = | Peripheral Cₘₐₓ = | Peripheral Cₘₐₓ = | Peripheral Cₘₐₓ = |
| 78 ng/mL | 2,405 ng/mL | below detection | below detection | 1,116 ng/mL |
| Peripheral | Peripheral AUCₗₐₛₜ = | Peripheral AUCₗₐₛₜ = | Peripheral | Peripheral AUCₗₐₛₜ = |
| AUCₗₐₛₜ = | 4,406 hr*ng/mL | below detection | AUCₗₐₛₜ = below | 3392 hr*ng/mL |
| 81 hr*ng/mL | | | detection | |

TABLE 6

| Compound I-13 | Niacin-linker Metabolite 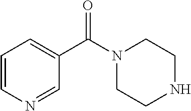 [SMILES: O=C(c1cccnc1)N1CCNCC1] | Linker-EPA Metabolite 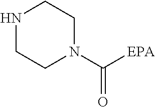 | Niacin 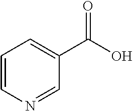 | Nicotinuric acid 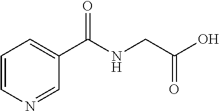 [SMILES: O=C(O)CNC(=O)c1cccnc1] |
|---|---|---|---|---|
| Portal Cₘₐₓ = | Portal Cₘₐₓ = 2,023 | Portal Cₘₐₓ = 36 | Portal Cₘₐₓ = 89 | Portal Cₘₐₓ = 34 |
| 6,068 ng/mL | ng/mL | ng/mL | ng/mL. | ng/mL |
| Portal AUCₗₐₛₜ = | Portal AUCₗₐₛₜ = | Portal AUCₗₐₛₜ = | Portal AUCₗₐₛₜ = | Portal AUCₗₐₛₜ = |
| 3,556 hr*ng/mL | 4,299 hr*ng/mL | 98 hr*ng/mL | 147 hr*ng/mL | 27 hr*ng/mL |
| Peripheral Cₘₐₓ = | Peripheral Cₘₐₓ = | Peripheral Cₘₐₓ = | Peripheral Cₘₐₓ = | Peripheral Cₘₐₓ = |
| 80 ng/mL | 1,441 ng/mL | 6 ng/mL | 71 ng/mL. | 5 ng/mL |
| Peripheral AUCₗₐₛₜ = | Peripheral AUCₗₐₛₜ = | Peripheral AUCₗₐₛₜ = | Peripheral | Peripheral AUCₗₐₛₜ = |
| 142 hr*ng/mL | 3,341 hr*ng/mL | 16 hr*ng/mL | AUCₗₐₛₜ = 33 | 18 hr*ng/mL |
| | | | hr*ng/mL | |

TABLE 7

| Compound I-23 | Niacin-linker Metabolite 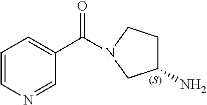 | Linker-EPA Metabolite 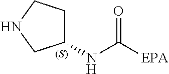 | Niacin 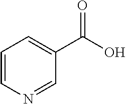 | Nicotinuric acid 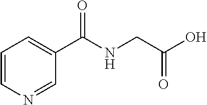 [SMILES: O=C(O)CNC(=O)c1cccnc1] |
|---|---|---|---|---|
| Portal Cₘₐₓ = | Portal Cₘₐₓ = 4,260 | Portal Cₘₐₓ = 7 | Portal Cₘₐₓ = | Portal Cₘₐₓ = |
| 2,604 ng/mL | ng/mL | ng/mL | 24ng/mL. | below detection |
| Portal AUCₗₐₛₜ = | Portal AUCₗₐₛₜ = | Portal AUCₗₐₛₜ = 5 | Portal AUCₗₐₛₜ = | Portal AUCₗₐₛₜ = |
| 1,436 hr*ng/mL | 8,636 hr*ng/mL | hr*ng/mL | 18 hr*ng/mL | below detection |
| Peripheral Cₘₐₓ = | Peripheral Cₘₐₓ = | Peripheral Cₘₐₓ = | Peripheral Cₘₐₓ = | Peripheral Cₘₐₓ = |
| 68 ng/mL | 2,864 ng/mL | below detection | below detection | 75 ng/mL |
| Peripheral AUCₗₐₛₜ = | Peripheral AUCₗₐₛₜ = | Peripheral AUCₗₐₛₜ = | Peripheral | Peripheral AUCₗₐₛₜ = |
| 60 hr*ng/mL | 6,420 hr*ng/mL | below detection | AUCₗₐₛₜ = below | 217 hr*ng/mL |
| | | | detection | |

TABLE 8

| Compound I-41 | Niacin-linker Metabolite 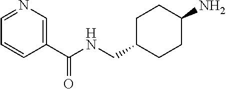 [SMILES: N[C@H]1CC[C@H](CNC(=O)c2cccnc2)CC1] | Linker-EPA Metabolite 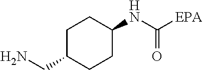 | Niacin 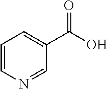 | Nicotinuric acid 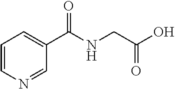 [SMILES: O=C(O)CNC(=O)c1cccnc1] |
|---|---|---|---|---|
| Portal Cₘₐₓ = | Portal Cₘₐₓ = 726 | Portal Cₘₐₓ = 2 | Portal Cₘₐₓ = 6 | Portal Cₘₐₓ = |
| 1,614 ng/mL | ng/mL | ng/mL | ng/mL. | below detection |
| Portal AUCₗₐₛₜ = | Portal AUCₗₐₛₜ = | Portal AUCₗₐₛₜ = 5 | Portal AUCₗₐₛₜ = | Portal AUCₗₐₛₜ = |
| 2,507 hr*ng/mL | 1,893 hr*ng/mL | hr*ng/mL | 48 hr*ng/mL | below detection |
| Peripheral Cₘₐₓ = | Peripheral Cₘₐₓ = | Peripheral Cₘₐₓ = | Peripheral Cₘₐₓ = | Peripheral Cₘₐₓ = |
| 109 ng/mL | 252 ng/mL | below detection | below detection | below detection |
| Peripheral | Peripheral AUCₗₐₛₜ = | Peripheral AUCₗₐₛₜ = | Peripheral | Peripheral AUCₗₐₛₜ = |
| AUCₗₐₛₜ = | 681 hr*ng/mL | below detection | AUCₗₐₛₜ = below | below detection |
| 218 hr*ng/mL | | | detection | |

### Example 13

### Analysis of Compound Hydrolysis and Tissue Distribution

A study was performed to determine the amount of test compound (e.g., compound I-15) and its metabolites in the liver of rats following oral administration of test compound for 3.5 days to achieve steady state. The amount of test compound and its metabolites in the liver provides information on distribution of such compounds within the rat, as well as information on stability of the test compound, such as resistance of the test compound to hydrolytic degradation. Experimental procedures and results are provided below.

### Part I—Procedures

A test compound was administered to a single group of Male Sprague-Dawley rats. Each group contained 6 animals. The test compound was administered by oral gavage at dose of 100 mg/kg in a vehicle consisting of Tween (specifically Tween 80), Peceol, PEG400 (at a ratio of 40:50:10) along with 0.2% tocopherol and emulsified in water, with a water to oil ratio of 66:1 (the dose concentration was 20 mg/mL and the dose volume was 5 mL/kg). Two additional animals were administered vehicle alone as controls.

Animals were dosed every 12 hours for 3.5 days (7 doses total). Rats were fasted overnight after the PM dose on day 3 of study. On day 4 the rats were euthanized 4 hours after the last dose. Vehicle treated rats were also fasted prior to collection of tissue samples on day 4. At 4 hours after last dose, the livers were excised, rinsed and weight of each tissue recorded. Livers were snap frozen in liquid nitrogen and stored in 50 mL polypropylene conical centrifuge tubes at −80° C.

Liver tissue was homogenized in PBS at a volume of 2 mL per gram of tissue. The amount of test compound and various metabolites of the test compound present in the liver homogenates were analyzed by LC/MS/MS as done in the previous example.

For a given test compound, the following compounds were quantitated from the collected homogenates: 1) test compound, 2) niacin linker, 3) linker-Fatty Acid, 4) niacin and 5) nicotinuric acid. In order to quantitate each of these compounds in the homogenates, standard curves in blank liver homogenates were generated using the appropriate internal standard.

### Part II—Results

Results from the study are provided in Table 9. The symbol “nd” means that the amount of analyte was not determined because the amount of any analyte present was below the level of detection in this assay.

TABLE 9

| | Amount of Analyte | | | | |
|---|---|---|---|---|---|
| | (nanograms of analyte per gram of tissue homogenate) | | | | |
| | Intact | Linker-Fatty | Niacin- | Nico- | |
| Test | Test | Acid | linker | tinuric | |
| Compound | Compound | Metabolite | Metabolite | Acid | Niacin |
| I-7 | 6.70 | 18.2 | 1753 | 275 | nd |
| I-8 | nd | nd | 2351 | 785 | nd |
| I-15 | 459 | 648 | 826 | 306 | nd |
| I-13 | 60.5 | 15782 | 2023 | 15.1 | nd |
| I-23 | nd | nd | 2290 | nd | nd |
| I-24 | 1072 | 77.2 | 2826 | nd | nd |

Compounds I-15 and I-24 had a significantly larger amount of intact test compound present in the liver than other compounds reported in Table 9. The data illustrate that the linker component of the fatty acid niacin conjugate has a profound impact on the amount of intact test compound present in the liver following oral administration of the test compound, and the amount of metabolites of said test compound that are present in the liver following oral administration of the test compound. The amount of intact test compound and the amount of metabolites of said test compound that are present in the liver provide information on the distribution of said compounds within the test subject, and provide information on the resistance of the test compound to hydrolytic degredation (i.e., hydrolysis).

Without being bound by a particular theory, it is understood that the significantly larger amount of intact test compounds I-15 and I-24 in the liver is attributable to, at least in part, greater absorption of intact compound into the portal circulation than other test compounds, such as compound I-8.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A compound having the formula: 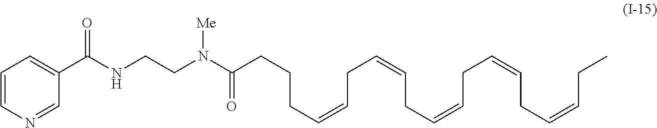 [SMILES: CC/C=C\C/C=C\C/C=C\C/C=C\C/C=C\CCCC(=O)N(C)CCNC(=O)c1cccnc1]
or a pharmaceutically acceptable salt thereof.

2. A compound having the formula: 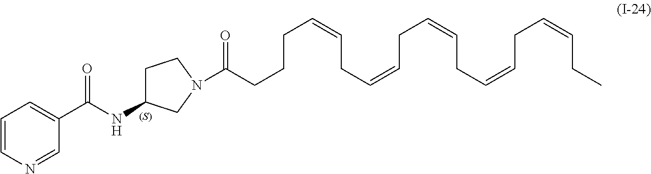
or a pharmaceutically acceptable salt thereof.

3. A pharmaceutical composition comprising the compound of claim 1 and a pharmaceutically acceptable carrier.

4. A pharmaceutical composition comprising the compound of claim 2 and a pharmaceutically acceptable carrier.

5. A method of treating or preventing a metabolic disease, comprising administering to a patient in need thereof an effective amount of a compound of claim 1 to treat or prevent the disease.

6. A method of treating or preventing a metabolic disease, comprising administering to a patient in need thereof an effective amount of a compound of claim 2 to treat or prevent the disease.

7. The method of claim 5, wherein the metabolic disease is atherosclerosis, dyslipidemia, coronary heart disease, hypercholesterolemia, cardiovascular disease, heterozygous familial hypercholesterolemia, homozygous familial hypercholesterolemia, fatty liver disease, nonalcoholic fatty liver disease (NFLD), nonalcoholic steatohepatitis (NASH), or fatty liver disease induced by treatment with a microsomal triglyceride transfer protein (MTP) inhibitor.

8. The method of claim 6, wherein the metabolic disease is atherosclerosis, dyslipidemia, coronary heart disease, hypercholesterolemia, cardiovascular disease, heterozygous familial hypercholesterolemia, homozygous familial hypercholesterolemia, fatty liver disease, nonalcoholic fatty liver disease (NFLD), nonalcoholic steatohepatitis (NASH), or fatty liver disease induced by treatment with a microsomal triglyceride transfer protein (MTP) inhibitor.

9. The method of claim 5 or 6, wherein the metabolic disease is hypertriglyceridemia, hypercholesterolemia, fatty liver disease, nonalcoholic steatohepatitis, or dyslipidemia.

10. The method of claim 5 or 6, wherein the metabolic disease is hypertriglyceridemia.

11. The method of claim 5 or 6, wherein the metabolic disease is hypercholesterimia.

12. The method of any one of claims 5-11, wherein the patient is treated with a statin.

13. The method of any one of claims 5-11, wherein the patient is treated with an MTP inhibitor.

14. The method of any one of claims 5-11, wherein the patient is treated with a Nieman Pick protein inhibitor.

15. The method of claim 14, wherein the Nieman Pick protein inhibitor is ezetimimide.

16. A method of treating a subject suffering from a disorder associated with hyperlipidemia and/or hypercholesterolemia, the method comprising administering to the subject in need thereof a SREBP inhibitor to provide an effective amount of the SREBP inhibitor in the subject's liver tissues to ameliorate the disorder, wherein said SREBP inhibitor is one of the following compounds or a pharmaceutically acceptable salt thereof:

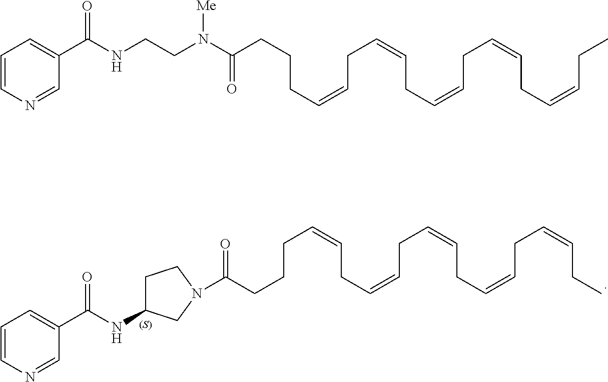

17. A pharmaceutical composition comprising a N-alkylated fatty acid niacin conjugate as an effective ingredient having accumulation in liver tissue and/or resistance to hydrolytic degradation, wherein the N-alkylated fatty acid niacin conjugate is one of the following or a pharmaceutically acceptable salt thereof:

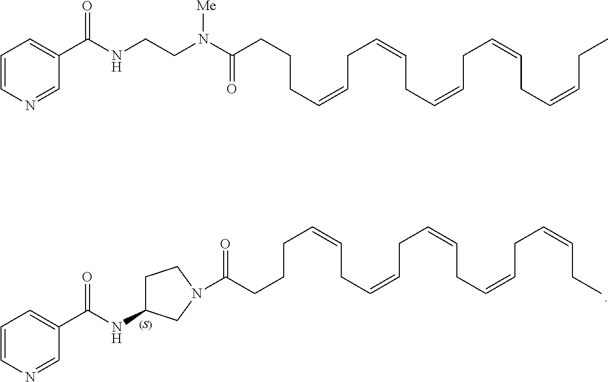

18. The pharmaceutical composition of claim 17, wherein the ingredient accumulates in liver tissue.

19. The pharmaceutical composition of claim 17, wherein the ingredient has resistance to hydrolytic degradation.

20. The pharmaceutical composition of claim 17, wherein the ingredient accumulates in liver tissue and has resistance to hydrolytic degradation.

21. The pharmaceutical composition of claim 17, 18, or 20, wherein a greater amount of the ingredient accumulates in liver tissue than in intestinal tissue.

22. The pharmaceutical composition of claim 17, 19, or 20, wherein resistance to hydrolytic degradation is resistance to hydrolytic degradation in the intestine.

23. The pharmaceutical composition of any one of claims 17-22, wherein the ingredient accumulates in the liver of a rat at an amount that is greater than twenty times the amount of the following compound that accumulates in the liver of a rat following oral administration of an equimolar amount of the following compound:

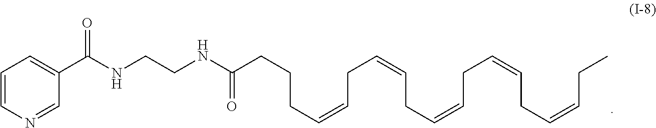 [SMILES: CC/C=C\C/C=C\C/C=C\C/C=C\C/C=C\CCCC(=O)NCCNC(=O)c1cccnc1]

24. The pharmaceutical composition of any one of claims 17-23, wherein the N-alkylated fatty acid niacin conjugate is 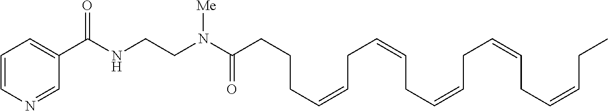 [SMILES: CC/C=C\C/C=C\C/C=C\C/C=C\C/C=C\CCCC(=O)N(C)CCNC(=O)c1cccnc1]
or a pharmaceutically acceptable salt thereof.

25. The pharmaceutical composition of any one of claims 17-23, wherein the N-alkylated fatty acid niacin conjugate is

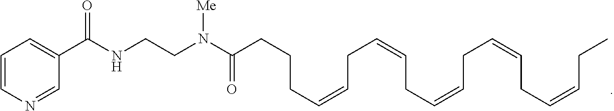 [SMILES: CC/C=C\C/C=C\C/C=C\C/C=C\C/C=C\CCCC(=O)N(C)CCNC(=O)c1cccnc1]

26. The pharmaceutical composition of any one of claims 17-23, wherein the N-alkylated fatty acid niacin conjugate is 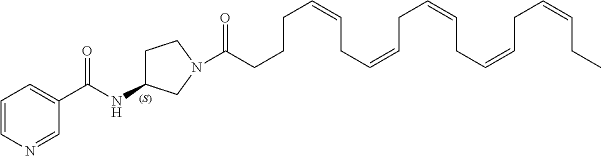
or a pharmaceutically acceptable salt thereof.

27. The pharmaceutical composition of any one of claims 17-23, wherein the N-alkylated fatty acid niacin conjugate is

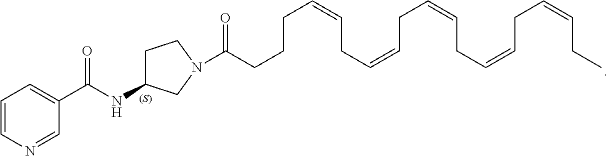

28. The pharmaceutical composition of any one of claims 17-27, wherein the pharmaceutical composition is for use in treating a metabolic disease.

29. The pharmaceutical composition of claim 28, wherein the metabolic disease is atherosclerosis, dyslipidemia, coronary heart disease, hypercholesterolemia, cardiovascular disease, heterozygous familial hypercholesterolemia, homozygous familial hypercholesterolemia, fatty liver disease, nonalcoholic fatty liver disease (NFLD), nonalcoholic steatohepatitis (NASH), or fatty liver disease induced by treatment with a microsomal triglyceride transfer protein (MTP) inhibitor.

30. The pharmaceutical composition of claim 28, wherein the metabolic disease is hypertriglyceridemia, hypercholesterolemia, fatty liver disease, nonalcoholic steatohepatitis, or dyslipidemia.

31. The pharmaceutical composition of claim 28, wherein the metabolic disease is hypertriglyceridemia.

32. Use of a N-alkylated fatty acid niacin conjugate in the manufacture of a pharmaceutical composition for the treatment of a metabolic disease, the N-alkylated fatty acid niacin conjugate being an effective ingredient having accumulation in liver tissue and/or resistance to hydrolytic degradation, wherein the N-alkylated fatty acid niacin conjugate is one of the following or a pharmaceutically acceptable salt thereof:

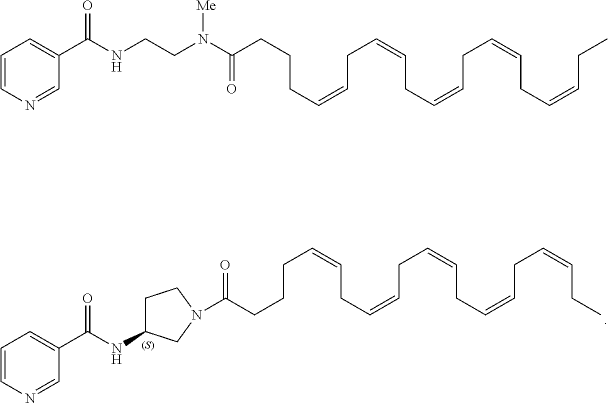

33. The use of claim 32, wherein the ingredient accumulates in liver tissue.

34. The use of claim 32, wherein the ingredient has resistance to hydrolytic degradation.

35. The use of claim 32, wherein the ingredient accumulates in liver tissue and has resistance to hydrolytic degradation.

36. The use of claim 32, 33, or 35, wherein a greater amount of the ingredient accumulates in liver tissue than in intestinal tissue.

37. The use of claim 32, 34, or 35, wherein resistance to hydrolytic degradation is resistance to hydrolytic degradation in the intestine.

38. The use of any one of claims 32-37, wherein the ingredient accumulates in the liver of a rat at an amount that is greater than twenty times the amount of the following compound that accumulates in the liver of a rat following oral administration of an equimolar amount of the following compound:

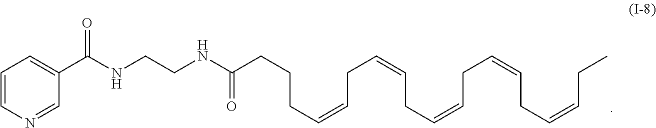 [SMILES: CC/C=C\C/C=C\C/C=C\C/C=C\C/C=C\CCCC(=O)NCCNC(=O)c1cccnc1]

39. The use of any one of claims 32-38, wherein the N-alkylated fatty acid niacin conjugate is 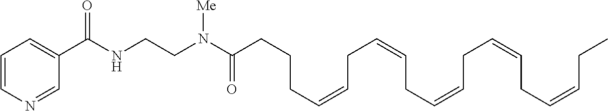 [SMILES: CC/C=C\C/C=C\C/C=C\C/C=C\C/C=C\CCCC(=O)N(C)CCNC(=O)c1cccnc1]
or a pharmaceutically acceptable salt thereof.

40. The use of any one of claims 32-38, wherein the N-alkylated fatty acid niacin conjugate is

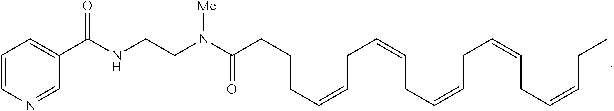 [SMILES: CC/C=C\C/C=C\C/C=C\C/C=C\C/C=C\CCCC(=O)N(C)CCNC(=O)c1cccnc1]

41. The use of any one of claims 32-38, wherein the N-alkylated fatty acid niacin conjugate is 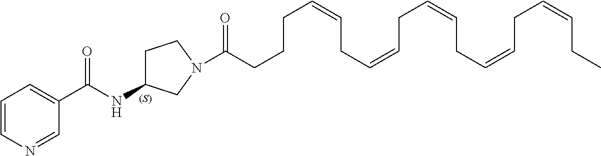
or a pharmaceutically acceptable salt thereof.

42. The use of any one of claims 32-38, wherein the N-alkylated fatty acid niacin conjugate is

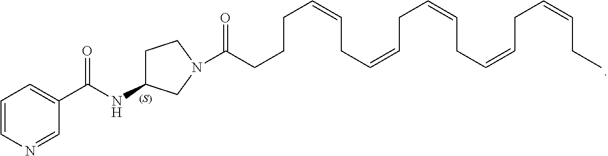

43. The use of any one of claims 32-42, wherein the metabolic disease is atherosclerosis, dyslipidemia, coronary heart disease, hypercholesterolemia, cardiovascular disease, heterozygous familial hypercholesterolemia, homozygous familial hypercholesterolemia, fatty liver disease, nonalcoholic fatty liver disease (NFLD), nonalcoholic steatohepatitis (NASH), or fatty liver disease induced by treatment with a microsomal triglyceride transfer protein (MTP) inhibitor.

44. The use of any one of claims 32-42, wherein the metabolic disease is hypertriglyceridemia, hypercholesterolemia, fatty liver disease, nonalcoholic steatohepatitis, or dyslipidemia.

45. The use of any one of claims 32-42, wherein the metabolic disease is hypertriglyceridemia.
